(19) United States
(12) Patent Application Publication
(10) Pub. No.: US 2026/0079158 A1
(43) Pub. Date: Mar. 19, 2026

(54) METHOD AND REAGENTS FOR ULTRASENSITIVE IMMUNOASSAY

(71) Applicant: Agri-Analysis, LLC, Davis, CA (US)

(72) Inventor: Ai-Ping Wei, Davis, CA (US)

(21) Appl. No.: 19/331,944

(22) Filed: Sep. 17, 2025

Related U.S. Application Data
(60) Provisional application No. 63/695,659, filed on Sep. 17, 2024.

(51) Int. Cl. G01N 33/543; C07K 14/195; C07K 16/10; C07K 16/12; C07K 16/30; C12Q 1/28; G01N 21/76; G01N 33/569; G01N 33/574

(52) U.S. Cl. CPC G01N 33/54388; C07K 14/195; C07K 16/104; C07K 16/1232; C07K 16/3069; C12Q 1/28; G01N 21/76; G01N 33/56916; G01N 33/56983; G01N 33/57555; C07K 2317/569; C07K 2319/61; G01N 2333/165; G01N 2333/245; G01N 2333/908

(57) ABSTRACT

Disclosed are assays, reagents, and methods for ultrasensitive detection of target molecules. The assay comprises fusion proteins including binding moieties, such as antibodies, nanobodies (VHH/VNAR), or aptamers, linked to nonfunctional fragments of a protein. In the presence of a target molecule, the binding moieties recognize distinct target regions and bring the protein fragments into close proximity, reconstituting a functional protein that generates a detectable signal. Linkers connecting the fusion components may be flexible peptides or polypeptides that spontaneously form dimers, trimers, or tetramers, thereby providing multivalent fusion proteins with enhanced sensitivity. The reconstituted protein may produce luminescent, fluorescent, colorimetric, or spectroscopic signals detectable by microplate readers, handheld luminometers, or lateral flow devices. The invention encompasses solid-phase, homogeneous, and lateral flow assay formats for detecting viruses, bacteria, proteins, peptides, or small molecules, including GLRaV-3, SARS-CoV-2, PSA, and E. coli. The disclosed assays exhibit improved specificity, reduced background, and enhanced signal-to-noise ratios.

### CROSS-REFERENCE TO RELATED APPLICATIONS

The application claims the benefit of 35 U.S.C. § 119(e) and under Article 8 of the Patent Cooperation Treaty to U.S. Provisional Patent Application 63/695,659 filed on Sep. 17, 2024, the contents of the entirety of which are incorporated herein by this reference.

### STATEMENT REGARDING ELECTRONIC FILING OF A SEQUENCE LISTING

The XML file named “Sequence Listing-P16313.1PC” created on Sep. 17, 2025, with a size of 81,501 bytes is hereby incorporated herein in its entirety by this reference.

### TECHNICAL FIELD

The application relates generally to diagnostics, prevention, and treatment of diseases, and, more particularly, to sensitive methods utilizing ultra-stable and ultrasensitive reagents for testing biological agents and diseases, and to methods and composition for the detection of diseases in central laboratories and/or in point-of-care settings that may be adapted into high-, medium- and low-throughput formats depending on specific application requirements.

### BACKGROUND

Since its invention by Yalow and Berson in 1960s, immunoassay methods have been crucial in modern science and medicine due to their ability to precisely detect and quantify specific biological molecules, such as proteins, hormones, and antibodies. These techniques rely on the highly specific interaction between antigens and antibodies, allowing for accurate measurement even at very low concentrations. Several FDA-approved immunoassay methods are widely used in clinical diagnostics and research. Some notable examples include: 1) Enzyme-Linked Immunosorbent Assay (ELISA), which is a widely used method that detects and quantifies proteins, hormones, and antibodies in a sample. ELISA is commonly employed for diagnosing conditions such as HIV, Lyme disease, and various autoimmune disorders. 2) Home Pregnancy Test, which is a specific ELISA that is sold over the counter to detect human chorionic gonadotropin (hCG) in urine, confirming pregnancy with high accuracy. 3) Chemiluminescent Immunoassays (CLIA), which utilize light emission for detecting the presence of specific analytes including those for cardiac biomarkers like troponin in low pg/ml concentration range to diagnose such conditions as heart attack and heart failure. 4) Radioimmunoassays (RIA) used for detecting hormones and drugs in blood samples. RIA relies on radiolabeled antibodies or antigens for quantification. 5) Lateral Flow Immunoassays (LFIA) are commonly used in point-of-care testing, such as rapid tests for detecting infections like influenza and COVID-19. LFIAs are designed for quick, on-site testing with easy-to-read results. By providing reliable data, immunoassays facilitate early detection of conditions, guide treatment decisions, and contribute to the development of new therapies, underscoring their pivotal role in advancing biomedical research and practices.

The Enzyme-Linked Immunosorbent Assay (ELISA) was developed in 1971 by Perlmann and Engvall. Their innovation involved using enzyme-linked antibodies to detect and quantify specific proteins or antigens in a sample, offering a more versatile and safer alternative to the radioactive methods used in immunoassays at the time. The ELISA process involves several steps: 1) immobilizing an antigen or antibody on a solid surface; 2) adding a sample; 3) adding a secondary antibody labeled with an enzyme; 4) adding a substrate that, upon the enzyme-substrate reaction, produces a detectable signal such as a change in color, fluorescence, bioluminescence or chemiluminescence, indicating the presence and concentration of the target analyte. A washing step is included in between each of foregoing steps. The advantages of ELISA include high sensitivity, specificity, versatility, ease of use, tolerance to interferents in sample matrix and is adaptable to both high throughput format (e.g., 96 or 384 wells) and single use test (e.g., home pregnancy test). This breakthrough technology enabled precise measurements of biological substances and has since evolved into various forms. Today, ELISA has become a fundamental tool widely utilized in a broad range of applications including clinical diagnostics, food safety, animal diagnostics, forensics, plant protection, environmental protection, and research. Despite advances in other testing technologies such as polymerase chain action (PCR) and mass spectrometry, ELISA remains a cornerstone of diagnostic and research laboratories because of its reliability, accuracy, ease of use, and low barrier to adoption.

While a valuable diagnostic tool, ELISA does come with certain limitations and disadvantages:
Cross-Reactivity and Specificity Issues: ELISA can sometimes produce false positives or false negatives due to cross-reactivity, where antibodies may bind to non-target antigens, or due to the presence of interfering substances in the sample. This is especially true when testing for closely related molecules, leading to false results.
Limited Dynamic Range: The dynamic range of ELISA can be limited, making it less suitable for detecting very high or very low concentrations of analytes. This might necessitate the use of dilution or alternative methods for accurate quantification.
Interference from Substances: The presence of certain substances in the sample, such as heterophilic antibodies or high levels of non-target proteins, can interfere with the assay's performance and lead to inaccurate results.
Time Consumption: The assay involves multiple steps, including coating, blocking, washing, sample incubation, secondary antibody incubation, substrate incubation steps, which can be time-consuming and require careful handling to avoid errors.

It is thus desirable to have a new ELISA assay that overcomes some of these disadvantages while maintaining and extending its advantages.

Enzyme fragment complementation assays (EFCs), also called split enzyme complementation, are a proven molecular tool for studying protein-protein interactions, especially for interrogating signal transduction processes. In EFC, an active enzyme molecule is split into two inactive fragments. One fragment is fused to the first protein of interest, and the other fragment is fused to a second interacting target of the first protein. Both fusion proteins are designed so be intracellularly expressed. When there is no interaction between the two proteins, the two enzyme fragments stay apart, no active enzyme is formed. When the cell is interrogated by a stimulus, the two proteins are activated to interact with each other, bringing the two enzyme fragments together. When in close proximity, the two fragments can recombine to form an active enzyme. Therefore, the protein-protein interaction can be monitored by measuring the enzyme activity. The EFC technique is well proven and has been used in solution, cell lysate, live cells as well as in animal models (Ozawa 2009; Olson 2007; Wong 2011; Anderson, 2012; Takakura 2012), the disclosures of each are incorporated herein by reference in their entirety.

β-galactosidase and firefly luciferase are two well-studied enzymes for this purpose. Other enzymes and proteins have also been used including dihydrofolate reductase, α-lactamase, green fluorescent proteins and ubiquitin. An earlier experiment utilizing a split firefly luciferase for studying protein-protein interactions was performed by Luker et al., the disclosure of which is incorporated in its entirety by this reference (Luker, et al., 2004). Specifically, the N-terminal and C-terminal fragments of luciferase were fused to rapamycin-binding domain (FRB) of the kinase mammalian target of rapamycin and FK506-binding protein 12 (FKBP), respectively. The addition of rapamycin (i.e., the stimuli) induced complexation of the FRB and FKBP fragments, thus bringing the N-terminal and C-terminal fragments together to reconstitute the active firefly luciferase.

Camelid-derived nanobodies, also known as VHHs (variable domain of heavy-chain antibodies), are a unique class of antibody fragments derived from the immune systems of camels, llamas, and alpacas. Unlike conventional antibodies, which consist of heavy and light chains, camelid antibodies feature a single heavy chain that includes a variable domain, the VHH. This single-domain structure makes VHHs exceptionally small, with a molecular weight of approximately 12-15 kDa (as compared to 150,000 of a human or mouse IgG), and highly stable, even under harsh conditions (such as high temperature and high percent chemical solvent solution). They possess several distinctive properties that make them highly valuable in various applications:
Single-Domain Structure: VHHs are composed of a single variable domain of a heavy-chain antibody. This single-domain structure contributes to their small size, typically around 12-15 kDa.
High Stability: VHHs are remarkably stable and can maintain their functional integrity under extreme conditions, such as high temperatures, varying pH levels, various solvent and proteolytic environments. This stability enhances their utility in harsh conditions.
Small Size: The small size of VHHs allows them to penetrate tissues more effectively than conventional antibodies. This property enables them to access and bind to targets that are difficult for larger antibodies to reach, such as small or hidden epitopes.
High Affinity and Specificity: VHHs retain the ability to bind to specific antigens with high affinity and specificity, like traditional antibodies. This precision makes them effective in targeting molecules or cells.
Ease of Production: VHHs can be produced in a variety of systems, including bacteria, yeast, and mammalian cells. They can be efficiently expressed and purified, often at a lower cost compared to traditional antibodies.
Modularity: VHHs can be easily engineered and modified to enhance their properties, such as affinity, stability, or therapeutic efficacy. They can be used as building blocks in creating multi-specific or multi-functional constructs.

Applications of VHHs include: 1) Therapeutics: Due to their small size and ability to bind to a wide range of targets with high specificity, VHHs are utilized in the development of targeted therapies for various diseases, including cancer, autoimmune disorders, and infectious diseases. They can be engineered to target specific cancer antigens or used as carriers for drug delivery. Currently, at least five VHH-derived drugs have been approved by the FDA and other regulatory agencies including Caplacizumab, which was the first FDA-approved VHH drug in February 2019 for the treatment of adults with acquired thrombotic thrombocytopenic purpura (aTTP), a rare and potentially life-threatening blood disorder; 2) Diagnostics: VHHs are employed in diagnostic assays and imaging due to their stability and ability to bind to specific biomarkers. They are used in various platforms, including ELISA and immunohistochemistry, for detecting and quantifying disease markers with high precision. 3) Research Tools: VHHs serve as valuable tools in molecular biology and biochemistry for studying protein interactions and functions. Their small size allows them to penetrate tissues and interact with targets that are challenging for conventional antibodies. 4) Therapeutic Antidotes: In cases of poisoning or toxin exposure, VHHs can be developed to neutralize specific toxins, offering a rapid and effective antidote. 5) Biotechnology: VHHs are used in protein purification and as affinity reagents in various biotechnological applications due to their high specificity and stability, which enhances the efficiency and yield of the purification process. Overall, the unique properties of camelid-derived nanobodies make them versatile and valuable assets in both clinical and research settings, offering innovative solutions to a range of challenges in medicine and biotechnology.

There have been a few reports in the literature of using VHH and split nano-luciferase for the homogenous one-step detection of small (a 2,4-D herbicide) and large (soluble epoxide hydrolase (she)) molecules and for (Sars-cov-2) antibodies, a result of the virus infection.

Ding et al. (2022) described a “ready-to-use” homogeneous competitive immunosensor for 2,4-dichlorophenoxyacetic acid (“2,4-D”), a common herbicide used to control broadleaf weeds in agriculture, lawns, and forests. The VHH to 2,4-D was first identified using standard molecular biology tools and fused to the small C-terminal fragment of nano-luciferase via a (GGGS)2 spacer. A peptidomimetic of 2,4-D was then identified from the phage display peptide library under six-round panning conditions. The peptidomimetic essentially serves as a peptide surrogate of 2,4-D for binding to the VHH of 2,4-D. For the immunosensor, the peptidomimetic is fused to the N-terminal end of nano-luciferase (i.e., the large fragment), via (GGGS)n (SEQ ID NO:21) spacers. The C-terminal fragment of nano-luciferase (i.e., small fragment) is fused, via (GGGS)n spacers, to the N-terminal end of the VHH. In the absence of 2,4-D, the peptidomimetic binds to VHH to bring the C- and N-terminal fragments of nano-luciferase to proximity so that they form a complex to reconstitute the active nano-luciferase. When 2,4-D is present, it competes with peptidomimetic for binding to VHH. Therefore, the bioluminescent signal therefrom is inversely proportional to the concentration of 2,4-D. This type of immunoassay is “ready-to-use” and has the advantages of label-free, immobilization-free and washing-free, and saves time and cost. After studying 24 fusion combinations with various spacers, the author reported signal-to-noise ratios varied between 2 to 41 (S/N). When the best combination was used, the assay sensitivity level of 3.64 ng/ml (IC50 value) was obtained for detection of 2,4-D. By comparison, assay sensitivity was reported to vary between 1.9-29.2 by other conventional assay methods for 2,4-D. The work of Ding et al. (2022) is homogenous assay conduced in solution with modest sensitivity and is for small molecule detection. Its similarity to the present invention is limited to the apparent use of VHH fusion with fragments of nano-luciferase. The publication of Ding et al. is incorporated herein in its entirety for reference.

He et al. (2023) described a “mix-and-read” homogeneous sandwich immunoassay for the detection of soluble epoxide hydrolase (sEH). sEH is a biomarker for and potential drug target of numerous diseases such as hypertension, chronic pain, various forms of cancer, and neurodegenerative conditions like Alzheimer's and Parkinson's disease. Several anti-sEH nanobodies were obtained using established molecular biology protocols for VHH and nanobody production. Selective anti-sEH nanobodies were individually fused with the N-terminal large fragment (LgLuc) and C-terminal small fragment (SmLuc) of nano-luciferase, respectively.

Different orientations of the LgLuc and SmLuc-nanobody fusions were expressed and investigated for their ability to reform the active NanoLuc in the presence of the sEH. After optimization, the assay demonstrated a high sensitivity of 1.4. ng/mL for human sEH, which is similar to that of conventional ELISA. The linear range of the assay could reach 3 orders of magnitude. The level of sensitivity was much higher than the clinical concentration range of mean fasting sEH level of 75.70 ng/mL with 95% confidence interval: 66.79 to 84.60 ng/mL. The publication of He et al. is incorporated herein in its entirety for reference.

Yao et al. (2021), the contents of which are incorporated herein by this reference, applied the split nano-luciferase concept for the detection of SARS-CoV-2 antibodies in. human serum post-infection. Instead of splitting firefly luciferase into an N-terminal (Nluc) and C-terminal (Cluc) fragment, Yao et al. split the nano-luciferase into three segments to mirror the FRB/FKBP/rapamycin interaction. The purpose of using three fragments instead of two was because the large fragment of nano-luciferase exhibits significant residual enzymatic activity and contribute to the high background noise, degrading the signal-to-noise ratio. In order to reduce background noise and improve signal-to-noise-ratio and therefore increase sensitivity, Yao et al. split nanoluciferase into three fragments and made two fusion proteins: (1) a β10 fragment fused to the ectodomain of the spike protein (S) of SARS-CoV-2 (β10-S), (2) a β9 fragment fused to Protein G (β9-G), and (3) a third and largest Δ11 S subunit lacking β9 and β10 that is not fused to any binding moiety and is present free in solution. When SARS-CoV-2 antibody is present the sample, it will bind to fusion proteins β10-S and β9-G simultaneously, which brings the and β9 and β10 fragments into proximity to each other to enable complexation with the Δ11S subunit in solution to reconstitute the bioluminescent activity of nanoluciferase.

The invention aims to improve upon the art by providing novel reagents and methods for ultrasensitive immunoassays to meet the diverse and demanding needs affecting life and health such as in disease diagnostics, food safety, agriculture production, public health, etc.

### SUMMARY OF THE DISCLOSURE

Disclosed are assays, methods, reagents, and fusion proteins for ultrasensitive detection of target molecules. The disclosure is based upon the principle of protein fragment complementation, in which non-functional fragments of a protein are recombinantly fused to binding moieties and brought into close proximity upon binding to a target molecule to reconstitute the protein. This reconstitution yields a functional protein that produces a detectable signal, enabling highly specific and sensitive assays for a wide range of applications.

The recombinantly fused molecule may include a functional linker (e.g., streptavidin or single-stranded DNA binding protein SSB) positioned in the N-, C-terminal or in-between the two subunits of the fusion molecule that will, upon spontaneous multimerization, render both the binding moiety and the protein fragment multivalent. It is reasoned that the multivalency will increase the binding affinity to the target, improve the complementation between the protein fragments and, therefore, increase the signal-to-noise ratio to surprisingly and unexpectedly provide assays of ultra sensitivity and specificity.

The binding molecules may include antibodies, antibody fragments, single-domain antibodies (VHHs or VNARs), and/or aptamers. The protein fragments may be portions of enzymes, fluorescent proteins, or related moieties capable of generating a measurable signal. Exemplary proteins include luciferase, nanoluciferase, β-galactosidase, β-lactamase, alkaline phosphatase, horseradish peroxidase, dihydrofolate reductase, glucose oxidase, ubiquitin, and fluorescent proteins. The reconstituted proteins may produce luminescent, fluorescent, colorimetric, or other spectral features measurable by optical instrumentation.

The disclosure also includes VHH21 (SEQ ID NO:22), VHH51 (SEQ ID NO:23), and VHH111 (SEQ ID NO:24) together or separately.

The disclosure encompasses various assay formats. In some embodiments, the assay is performed in a solid-phase format (e.g., an ELISA) using immobilized capture antibodies or nanobodies. In other embodiments, the assay is conducted in a homogeneous “mix-and-read” format without immobilization, labeling, or washing steps. In yet other embodiments, the assay is embodied in a lateral flow immunoassay, in which assay reagents are incorporated into a membrane strip for rapid point-of-care detection. The signal can be detected visually or by instruments such as a handheld luminometer, microplate reader, or lateral flow reader.

The assays are suitable for detecting a wide range of target molecules including viruses, bacteria, proteins, peptides, and small molecules. Specific examples include grapevine leafroll-associated virus type 3 (GLRaV-3), grapevine fanleaf virus (GVFL), SARS-CoV-2 nucleocapsid protein, human prostate-specific antigen (PSA), and bacterial pathogens such as Escherichia coli O157, Salmonella spp., and Listeria spp.

Retrofitted Diagnostic Formats Using Thermophile-Derived Enzyme Linkers: In additional embodiments, the disclosure may be applied by retrofitting conventional immunoassays such as ELISA or lateral flow tests to incorporate the split enzyme fusion proteins described herein. In a conventional ELISA, the detection antibody is conjugated to an enzyme label (e.g., alkaline phosphatase, horseradish peroxidase) that remains catalytically active irrespective of target binding, thereby contributing to nonspecific background. By contrast, in the present disclosure, the enzyme fragments are inactive until brought into proximity by binding of at least two detection molecules to a target. This substantially reduces background, improves signal-to-noise ratio, and enhances sensitivity.

For example, in an assay for prostate-specific antigen (PSA), a capture antibody is immobilized on a solid surface, and patient serum is added. Instead of a conventional single enzyme-labeled detection antibody, two detection reagents are employed: (i) a first VHH (VHHP33 (SEQ ID NO:31)) specific for a first PSA epitope fused via a flexible spacer and a thermophilic linker (e.g., SSB from T. aquaticus) to an N-terminal fragment of nanoluciferase, and (ii) a second VHH (VHHP53 (SEQ ID NO:32)) specific for a second PSA epitope fused via a thermophilic linker (e.g., SSB from T. thermophilus) to a C-terminal fragment of nanoluciferase. In the presence of PSA, the VHHs bind distinct epitopes, the thermophile-derived linkers multimerize, and the nanoluciferase fragments complement to form an active enzyme. Signal is generated only in the presence of target, reducing washing requirements and enhancing sensitivity.

Human Pathogen Detection: In some embodiments, the disclosure will be used for detection of viral antigens such as SARS-CoV-2 nucleocapsid protein. A pair of VHHs specific for non-overlapping epitopes of the N protein (e.g., VHHN4 (SEQ ID NO:27), VHHN6 (SEQ ID NO:28)) may be fused, via peptide spacers and thermophilic SSB linkers, to N- and C-terminal nanoluciferase fragments. In the presence of the N protein, the fusion proteins assemble into an active luciferase detectable in the presence of furimazine substrate. Because thermophilic linkers maintain stability at elevated temperatures (45-60° C.), such reagents are suitable for point-of-care settings with minimal refrigeration.

Plant Pathogen Detection: In further embodiments, the disclosure may be applied to agricultural viruses, such as grapevine leafroll-associated virus 3 (GLRaV-3). A first VHH (VHHF1 (SEQ ID NO:29)) recognizing a first epitope of the capsid protein and a second VHH (VHHF7 (SEQ ID NO:30)) recognizing a distinct epitope are fused, via thermophile-derived linkers, to complementary fragments of nanoluciferase. In the presence of virus particles, the fragments assemble into active enzyme, producing a luminescent signal detectable in field-deployable assays.

Bacterial Pathogen Detection: In additional embodiments, the disclosure iss applied to bacterial pathogens, such as E. coli O157:H7. VHHs specific to bacterial outer membrane proteins may be fused to nanoluciferase fragments with thermophile-derived linkers, permitting detection of live or lysed bacterial cells in food samples. These assays enable ultrasensitive bacterial detection within minutes and remain stable under variable food testing conditions.

### BRIEF DESCRIPTION OF THE DRAWINGS

FIG. 1 is a schematic of an assay and method according to embodiments of the disclosure where the first binding moiety 110 is connected, via a polypeptide linker 150, to the first fragment of a protein 130 to form a first fusion molecule 102, and the second binding moiety 120 is connected, via a polypeptide linker 150, to the second fragment of the protein 140 to form a second fusion molecule 104. When the first binding moiety 110 is recognized by and bound to the first region of the target molecule 100 (site A) and the second binding moiety 120 is recognized by and bound to the second region of the target molecule 100 (site B), the first fragment 130 and second fragment 140 of the protein are brought to close proximity to complex with each other so as to reconstitute the full function of protein such as enzymatic activity or fluorescence emission. The presence of the target molecule 100 is, therefore, detected by measuring the signal produced by the functional protein 160 relative to a negative control.

FIG. 2 is a schematic of an assay and method according to embodiments of the disclosure. In this embodiment, a polypeptide linker 150 is used between the first binding moiety 110 (e.g., VHH1) and first protein fragment 130 (e.g., N-terminal fragment), and between the second binding moiety 120 (e.g., VHH2) and second protein fragment 140 (e.g., N-terminal fragment), wherein the polypeptide linker 150 is a member of the protein family that spontaneously forms dimers, trimers, tetramers or multimers such as alkaline phosphatase, streptavidin, and single-stranded DNA binding proteins (SSB). A first spacer 115 connects the first binding moiety 110 to the polypeptide linker 150, and a second spacer 115 connects the second binding moiety 120 to the polypeptide linker 150. The incorporation of this type of polypeptide linker 150 represents a significant improvement upon the state of the art. The polypeptide not only serves as a linker to connect the binding moieties with the protein fragments but also renders the first and second fusion proteins 102 and 104 with divalent, trivalent, tetravalent or multivalent properties. These properties can significantly improve binding affinity, signal-to-noise, assay specificity and sensitivity. It is important to note that the first fusion molecule 102 of one structural arrangement may be combined with a second first fusion molecule 102 of the same structural arrangement or different structural arrangement to obtain the optimal results.

FIG. 3 is a schematic of a first fusion protein 102 comprising of first binding moiety 110, a polypeptide linker 150, a first portion of a protein 130, and a second fusion protein 104 comprising of a second binding moiety 120, a polypeptide linker 150, and a second portion of the protein 140, according to embodiments of the disclosure. The sequential order of connection among the first binding moiety 110, a polypeptide linker 150, a first portion of the protein 130 in the first fusion protein 102 and that of the second binding moiety 120, a polypeptide linker 150, a second portion 140 in the second fusion protein 104 are for illustrative purposes only and are not limited to that shown in the drawing. The optimal connection order depends upon the choice of the first and second binding moieties that are dictated by the target molecule 100 and can vary from assay to assay.

FIG. 4 is a schematic of a specific embodiment of assay and method according to the drawing shown in FIG. 1, where the functional protein 160 is nano-luciferase, first binding moiety 110 is a VHH (i.e., VHH1) specific for grapevine leafroll-associated virus type 3, first region (site A) is an epitope of GLRaV-3 capsid protein, and where first binding moiety 110 is another VHH (i.e., VHH2) specific for grapevine leafroll-associated virus type 3, second region (site B) is another epitope of GLRaV-3 capsid protein.

FIG. 5 is a schematic of specific embodiment of assay and method according to the drawing shown in FIG. 2, where the functional protein 160 is nano-luciferase, first binding moiety 110 is a VHH (i.e., VHH1) specific for grapevine leafroll-associated virus type 3 (GLRaV-3), first region (site A) is an epitope of GLRaV-3 capsid protein, and where the second binding moiety 120 is another VHH (i.e., VHH2) specific for GLRaV-3, second region (site B) is another epitope of GLRaV-3 capsid protein. The polypeptide linker 150 is streptavidin that spontaneously form tetramers so that VHH1, VHH2 and luciferase fragments are tetravalent.

FIG. 6 is schematics to compare conventional ELISA assay with the assay utilizing the methods and reagents of the disclosure where the assay utilizing the methods and reagents herein is conducted on solid surfaces such as the bottom of 96-well plates. Comparing to the conventional ELISA assay, assays utilizing the methods and reagents of the disclosure provide significant improvements upon the conventional ELISA assay in signal-to-noise ratio, assay sensitivity and specificity, wider dynamic range, assay speed, ease of use, and low assay cost.

FIG. 7 is a schematic of a “mix and read” assay utilizing the methods and reagents of the disclosure where the assay is conducted in solution phase where the presence of the target molecule 100 in a sample, upon binding with detection antibody VHH1 and detection antibody VHH2, can bring the enzyme fragments together to reconstitute the active enzyme. Readout of the enzymatic signal in the presence of its substrate can help determine the presence or absence of the target molecule 100 or quantitate the concentration the target molecule 100. The “mix and read” assay utilizing the methods and reagents of the disclosure provide significant improvement in signal-to-noise ratio, wider dynamic range, assay speed, ease of use, and low assay cost.

FIG. 8 is schematics to compare conventional lateral flow immunoassay (LFIA) with the new LFIA utilizing the methods and reagents of the disclosure where the assay is conducted on lateral flow strips comprising control line 175, test line 180, and sample and reagent pads 190. Comparing to the conventional LFIA assay, assays utilizing the methods and reagents of the disclosure provide significant improvements upon the conventional LFIA assay in signal-to-noise ratio, assay sensitivity and specificity, wider dynamic range and ability to quantitate while maintaining advantages of fast assay speed, ease of use, and low cost and ease of manufacturing.

FIG. 9 is a set of data showing experimental results using the method and reagents disclosed in this invention as illustrated in FIG. 2 for the detection of grapevine leafroll associated virus 3 (GLRaV-3). Specifically, FUSION 10, 11, 12 represent three fusion proteins each of which is between a VHH specific to grapevine leafroll associated virus 3 (GLRaV-3) designated as VHH21, VHH51 and VHH111 fused, via a (GGGSS)n (SEQ ID NO:2) linker, to N-terminal fragment of the nano-luciferase (Large Luc), respectively. Similarly, FUSION 7, 8 and 9 represent three different fusion proteins each of which is between a GLRaV-3 specific VHH (e.g., VHH21, VHH51 and VHH111) fused, via a (GGGSS)n (SEQ ID NO:2) linker, to the C-terminal fragment of Nano-luciferase (Small Luc, SmLuc), respectively. Of the nine (9) possible combinations, the combination between Fusion 11 (VHH51-GGGSSGG-LgLuc) (SEQ ID NO:18) with Fusion 7 (VHH21-GGGSSGGG-SmLuc) (SEQ ID NO:14) demonstrated the highest signal-to-noise ratio of approximately 122. Positive luminescence readings were from experiments using crude phloem tissue extracts of grapevine cuttings infected by GLRaV-3 virus. Negative control readings from experiments using crude phloem tissue extracts of grapevine cuttings free of GLRaV-3 infection.

FIG. 10 is a set of data showing experimental results using the method and reagents disclosed in this invention as illustrated in FIG. 2 for the detection of grapevine leafroll associated virus 3 (GLRaV-3). Specifically, FUSION 4, 5, 6 represent three different fusion proteins each of which is between a VHH specific to grapevine leafroll associated virus 3 (GLRaV-3) designated as VHH21, VHH51 and VHH111 fused, via a polypeptide linker 150, e.g., Streptavidin that spontaneously forms tetramers, to the N-terminal fragment of the Nano-luciferase (Large Luc, LgLuc), respectively. Similarly, FUSION 1, 2 and 3 represent three fusion proteins each of which is between a GLRaV-3 specific VHH (e.g., VHH21, VHH51 and VHH111) fused, via a polypeptide linker 150, e.g., Streptavidin that spontaneously forms tetramers, to the C-terminal fragment of Nano-luciferase (Small Luc, SmLuc), respectively. Of the nine (9) possible combinations, the combination between Fusion 6 (VHH111-streptavidin-lgLuc) with Fusion 1 (VHH21-streptavidin-SmLuc) demonstrated the highest signal-to-noise ratio of approximately 2280. Positive luminescence readings were from experiments using crude phloem tissue extracts of grapevine cuttings infected by GLRaV-3 virus. Negative control readings from experiments using crude phloem tissue extracts of grapevine cuttings free of GLRaV-3 infection.

FIG. 11 is a set of data showing experimental results using the method and reagents disclosed in this invention as illustrated in FIG. 2 for the detection of grapevine leafroll associated virus 3 (GLRaV-3). Specifically, FUSION 4, 5, 6 represent three different fusion proteins each of which is between a VHH specific to grapevine leafroll associated virus 3 (GLRaV-3) designated as VHH21, VHH51 and VHH111 fused, via a polypeptide linker 150, e.g., Streptavidin that spontaneously forms tetramers, to the N-terminal fragment of the Nano-luciferase (Large Luc, lgLuc), respectively. Similarly, FUSION 7, 8 and 9 represent three fusion proteins each of which is between a GLRaV-3 specific VHH (e.g., VHH21, VHH51 and VHH111) fused, via a (GGGSS)n linker (SEQ ID NO:2), to the C-terminal fragment of Nano-luciferase (Small Luc, SmLuc), respectively. Of the nine (9) possible combinations, the combination between Fusion 4 (VHH21-streptavidin-lgLuc) with Fusion 8 (VHH51-streptavidin-SmLuc) demonstrated the highest signal-to-noise ratio of approximately 490. Positive luminescence readings were from experiments using crude phloem tissue extracts of grapevine cuttings infected by GLRaV-3 virus. Negative control readings from experiments using crude phloem tissue extracts of grapevine cuttings free of GLRaV-3 infection.

FIG. 12 is a set of data showing experimental results using the method and reagents disclosed in this disclosure as illustrated in FIG. 2 for detecting grapevine leafroll associated virus 3 (GLRaV-3). Specifically, FUSION 10, 11, 12 represent three fusion proteins between three VHHs specific to GLRaV-3 designated as VHH21, VHH51 and VHH111 fused, via a (GGGSS)n linker, to N-terminal fragment of the Nano-luciferase (Large Luc, lgLuc), respectively. Similarly, FUSION 1, 2 and 3 represent three fusion proteins between the GLRaV-3 specific VHH21, VHH51 and VHH111 fused, via a polypeptide linker 150, e.g., Streptavidin that spontaneously forms tetramers, to the C-terminal fragment of Nano-luciferase (Small Luc, SmLuc), respectively. Of the nine (9) possible combinations, the combination between Fusion 10 (VHH111-GGGSSGG-lgLuc) (SEQ ID NO:17) with Fusion 3 (VHH21-streptavidin-SmLuc) demonstrated the highest signal-to-noise ratio of approximately 233. Positive luminescence readings were from experiments using crude phloem tissue extracts of grapevine cuttings infected by GLRaV-3 virus. Negative control readings from experiments using crude phloem tissue extracts of grapevine cuttings free of GLRaV-3 infection.

FIG. 13 is a graph showing results using the methods and reagents described herein for mix-and-read detection of GLRaV-3 in ten (10) grapevine cuttings. Phloem tissue chips (5×5 mm) from ten different grapevine cuttings were first added to ten different wells on a 96-well plate. Each phloem chip is taken using a sterile blade from under the lignified tissue and added directly to the well without touching anything besides the blade. Each grapevine cutting was from a different vine. A 1:1 mixture of the supernatant from the E. coli overnight culture of fusion proteins 1 and 6 (from Example 3), together with the appropriate amount of nano-luciferase substrate (Promega Corp), was added to the ten (10) wells containing the phloem tissue chips. Luminescence signal was read on a GlowMax luminometer (Promega Corp). Cutting 2 showed a signal level of 121-fold higher than the rest of the nine cuttings, indicating its positive status. RT-PCR testing of the same set of samples confirmed this result.

FIGS. 14A-14C are graphs showing results using the methods and reagents described herein for detecting GLRaV-3 in nine (9) serially diluted crude grapevine phloem samples known to be positive for GLRaV-3, plus a negative control. The signal starts to become indistinguishable from the negative control after 6561× dilution (FIG. 14A). By comparison, when the same set of serially diluted samples were tested using conventional ELISA methods, the signal starts to become indistinguishable from the negative control after 27× dilution (FIG. 14B was for colorimetric substrate, and FIG. 14C was for luminescent substrate). A sensitivity enhancement of approximately 250-fold was obtained in this experiment relative to conventional ELISA methods.

FIG. 15 shows the CT values of qPCR testing for GLRaV-3 as a function of dilution factor for the same set of serially diluted crude samples as used in FIGS. 14A-14C. The CT values become indistinguishable from the background after 81× dilution. Similar results were obtained when RT-PCR was used to test the same set of serially diluted samples (data not shown). A sensitivity improvement of approximately 81-fold was obtained in this experiment relative to qPCR or RT-PCR.

### DETAILED DESCRIPTION

The disclosure is directed to a broadly applicable assay platform rather than a single construct or sequence. The concept resides in the modular architecture, which employs: (i) at least two binding moieties directed to distinct regions of a target molecule; (ii) fragments of a functional protein that are individually inactive but reconstitute activity when brought into proximity by the binding moieties; and (iii) optional linker or spacer elements that provide flexibility, stability, or multivalency.

Importantly, the disclosure is not limited to any one sequence of a binding moiety, protein fragment, or linker. Rather, the disclosure teaches a general framework that may be implemented using a wide variety of known or yet-to-be-developed components:

Binding Moieties. These may include, without limitation, antibodies, antibody fragments, nanobodies (VHH/VNAR), aptamers, peptides, or small molecules. Any binding element capable of specifically recognizing a target epitope may be incorporated.

Protein Fragments. Suitable proteins include luciferases (e.g., firefly luciferase, nanoluciferase), β-galactosidase, alkaline phosphatase, horseradish peroxidase, fluorescent proteins, or other enzymes and signal-generating proteins. The invention is not tied to a particular amino acid sequence, but to the principle of using complementary fragments that only regain activity when colocalized.

Linkers/Spacers. Both simple flexible linkers (e.g., glycine-serine repeats) and functional polypeptides (e.g., streptavidin, alkaline phosphatase, single-stranded DNA binding proteins) may be used. The purpose of such linkers is to enhance binding orientation, valency, and overall assay performance.

This modularity enables the platform to be readily adapted for detecting any suitable target molecule, including viral proteins, bacterial antigens, human biomarkers, or plant pathogens. The examples provided herein (e.g., GLRaV-3, SARS-CoV-2, PSA) are illustrative and not limiting.

Accordingly, the disclosure is a generalizable platform that can be tailored to diverse applications by substituting appropriate binding moieties, protein fragments, and linkers without departing from the scope of the disclosure.

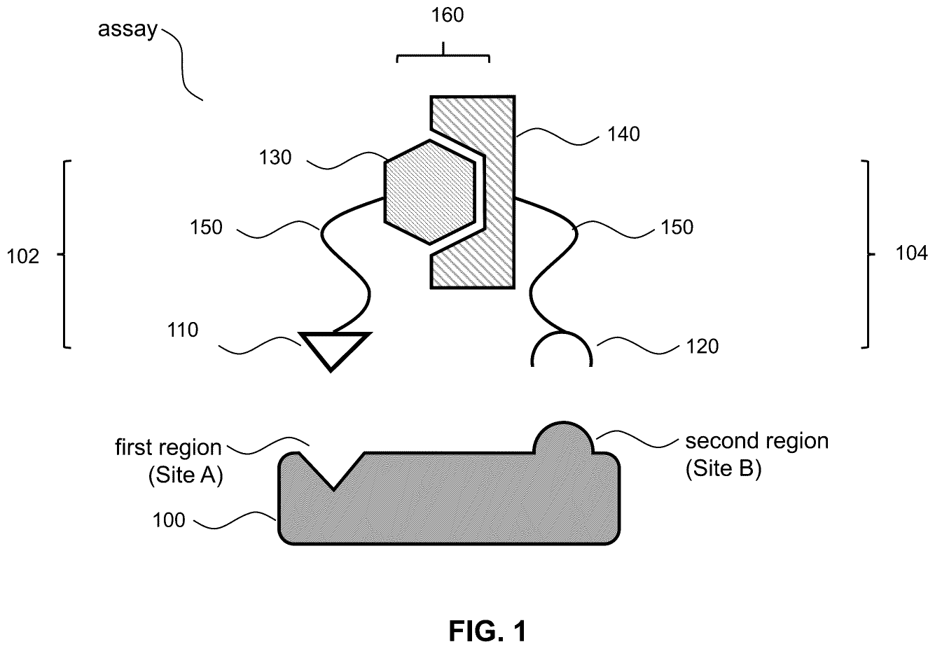

As, for example, illustrated in FIG. 1, described herein is an assay comprising of methods and reagents for the detection of a target molecule 100 such as a molecule, a virus and a bacterium, etc. The assay utilizes the target as the “bridging molecule” to ultimately bring protein fragments 130 and 140 together to restore protein activity (e.g., produce a photochemical response upon a catalytic reaction). The target molecule 100 may be a virus such as GLRaV-3, which is illustrated herein as an example. Other examples include the capsid protein of grapevine fanleaf virus, human COVID-19 virus, nucleocapsid (N) and spike (S) protein of COVID-19 virus, human prostate antigen (PSA), and human soluble epoxide hydrolase (HsEH), etc.

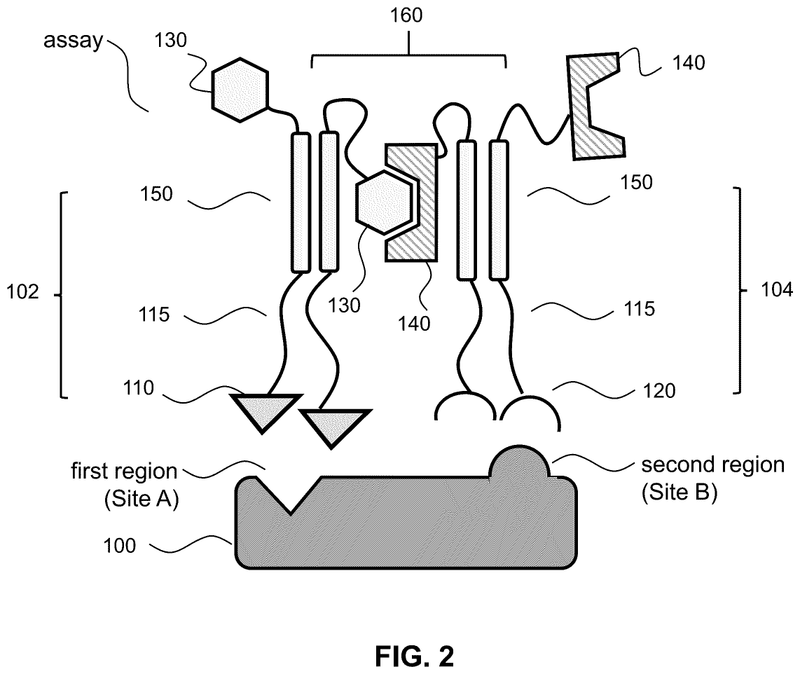

As illustrated in FIG. 2, the methods and reagents described herein further are comprised of two VHHs as binding moieties 110 and 120 and nano-luciferase (luc) as the moiety for signal generation. The VHHs are fused, via polypeptide linkers 150, to the fragments of nano-luciferase 130 and 140 to create VHH-Luc fragment fusion proteins that act together as a molecular sensor that has both recognition and signal amplification elements. Without the target molecule 100, the enzyme fragments 130 and 140 are separated, and no active enzyme 160 is formed, and no signal is produced. When the target molecule 100 is present, the virus-specific VHHs 110 and 120 will bind to the target molecule 100, bringing the enzyme fragments 130 and 140 into proximity to form an active luciferase enzyme 160. When using nanoluciferase, bioluminescence can be conveniently measured in the presence of its substrate such as furimazine by a handheld luminometer at point-of-use or by automated instruments configured for high throughput screening in a laboratory.

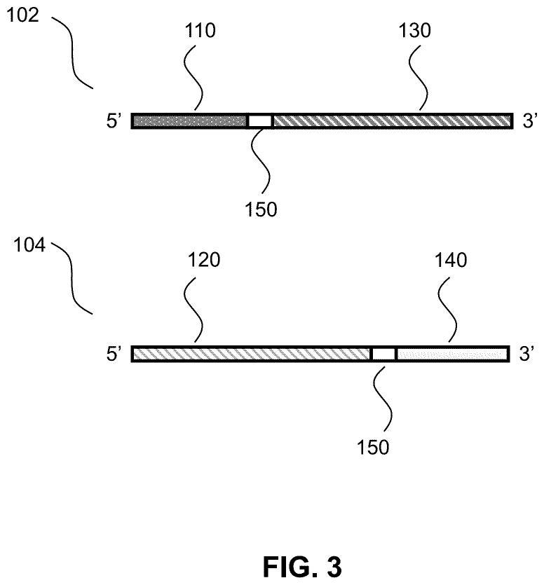

As illustrated in FIG. 2 and FIG. 3, the choice of polypeptide linkers 150 between the VHH and luciferase (luc) fragments is critical and most innovative part of this invention, wherein the polypeptide linker 150 may be a simple flexible linker such as (GGGSSGGG)n (SEQ ID NO:1), where n is an integer within a range of from 1 to 10, such as from 1-4, 5-7, or 8-10, or a polypeptide chain with known or unknown functions, and a combination thereof. The polypeptide linker 150 is judiciously chosen so that it spontaneously forms dimers, trimers, tetramers, and multimers so that VHH and luciferase (luc) fragments are multimeric when fused via such polypeptide linkers 150. The spontaneous multimerization of the polypeptide linker 150 will create dimers, trimers, tetramers or multimers of VHH and a luc fragment, which can provide increased affinity, specificity, sensitivity and better complementation between the two luc fragments compared to monomeric VHH and monomeric luc fragments.

The “linker” as defined in this invention is a functional protein such as streptavidin. It does not necessarily have to be positioned in-between a VHH and a nanoluciferase fragment (Luc). The polypeptide linker 150 may be positioned in the N-terminal, C-terminal or the middle of the fusion protein in the order of Linker-VHH-LgLuc, VHH-LgLuc-Linker or VHH-Linker-LgLuc; Linker-VHH-SmLuc, VHH-SmLuc-Linker or VHH-Linker-SmLuc.

There are “spacers” 115 between different subunits (the polypeptide linker 150, VHH 110/120 and a nanoluciferase fragment 130/140), which are hydrophilic and flexible peptide sequence such as (GGGSSGGG)n (SEQ ID NO:1), where n is an integer within a range of from 1 to 10. In certain constructs where the polypeptide linker 150 is not present, only spacers 115 are necessary to connect between different subunits.

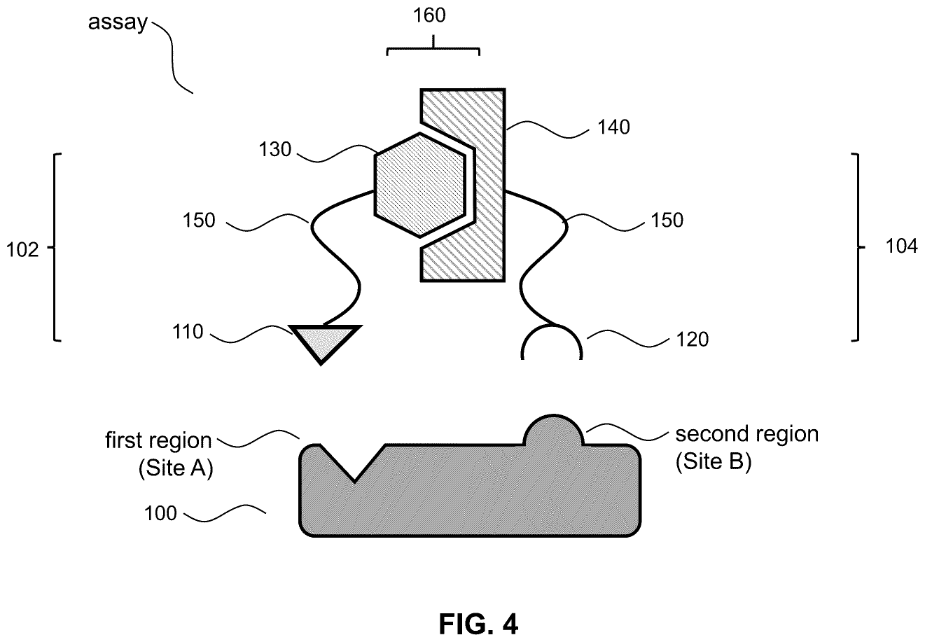

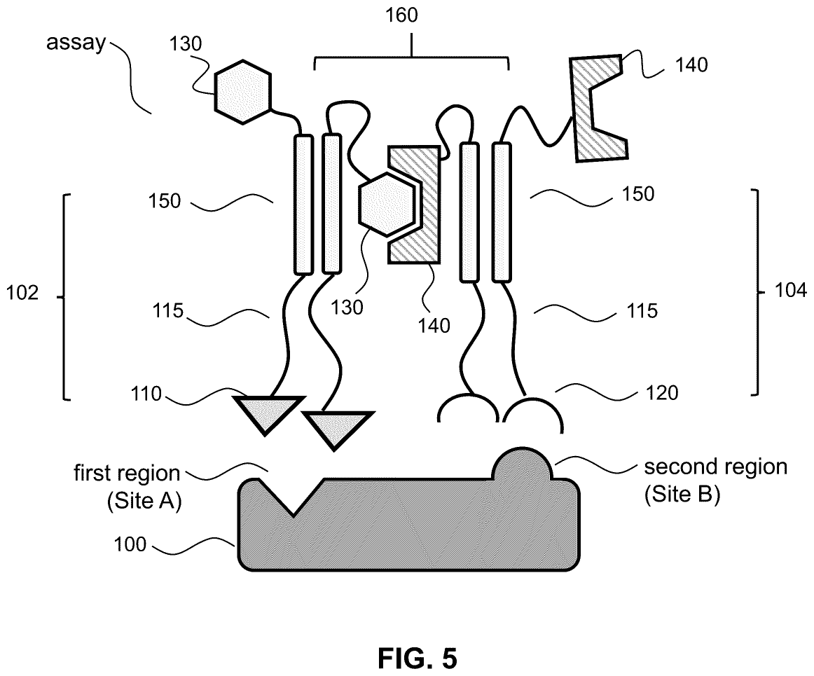

As illustrated in FIG. 4 and FIG. 5 the methods and reagents described herein are embodied for the early and rapid diagnosis and preventative screening of grapevine leafroll associated virus 3 (GLRaV-3), which is the number one viral pathogen affecting grape production worldwide.

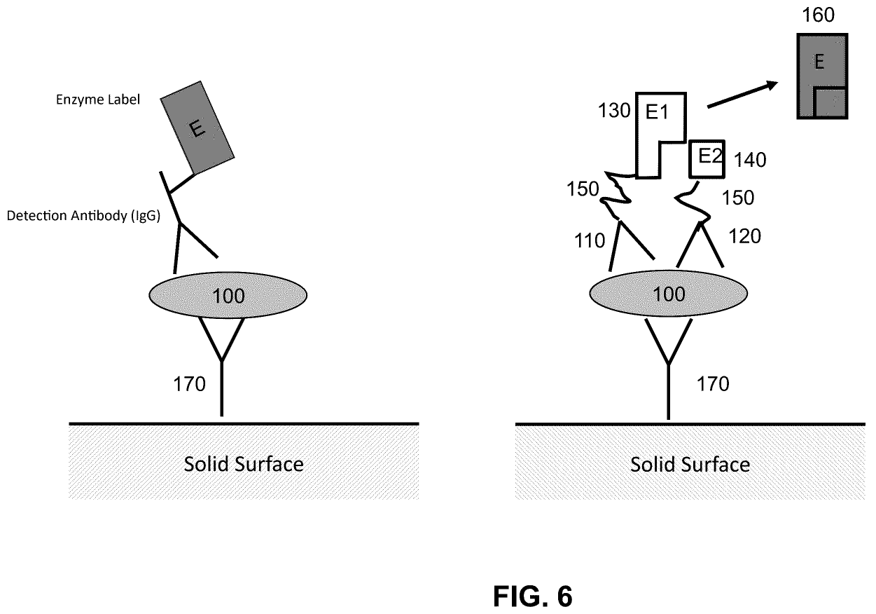

As illustrated in FIG. 6, the methods and reagents described herein can be embodied for the improvement of the conventional enzyme linked immunosorbent assay (ELISA). In this embodiment, the assay shares a similar first step where a capture antibody 170 (polyclonal, monoclonal or VHH) is coated onto the solid surface such as the bottom of 96-well plates. In the conventional ELISA, the enzyme label is always active before and after binding to the target molecule 100. When it adheres to the surface nonspecifically, the enzyme is still active and contributes nonspecific background noise, therefore reducing signal-to-noise ratio and assay sensitivity. By contrast, when using the methods and reagents described here, the enzyme is not activated until after the two binding moieties 110 and 120 simultaneously bind to their target molecule 100. This invention significantly improves the art in serval ways. First, the blocking step is unnecessary and eliminated, which can reduce time and cost. Second, sensitivity is significantly increased due to increase in signal-to-noise ratio. Third, nonspecific interference is significantly reduced because it requires two detection antibodies to create a signal as opposed one.

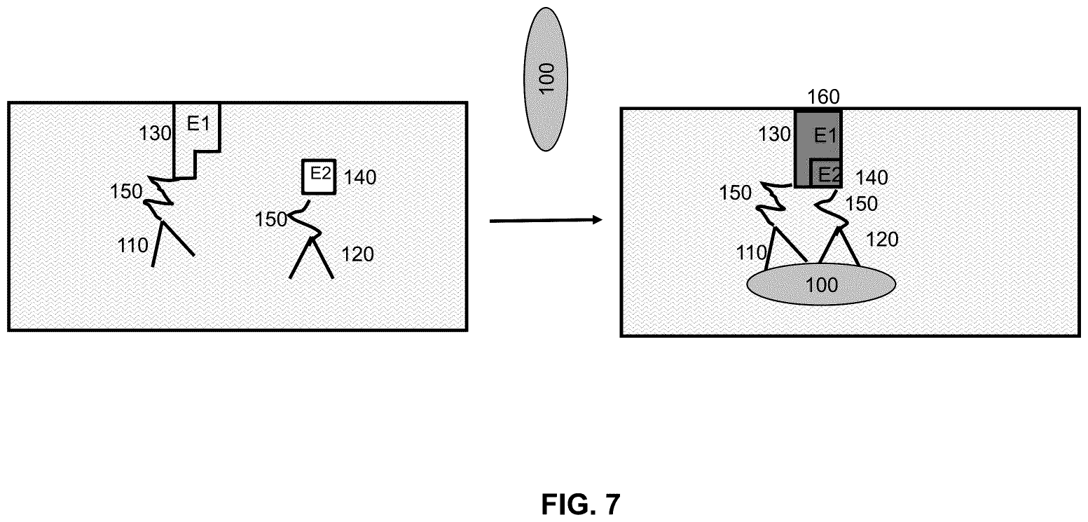

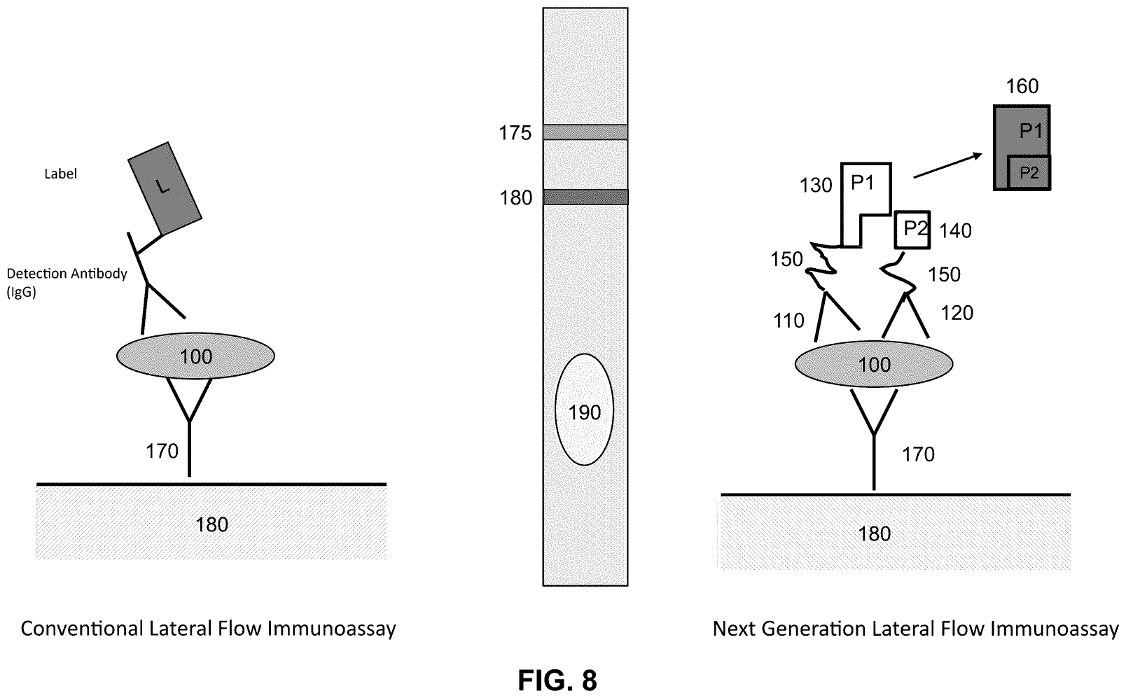

As illustrated in FIG. 7, the methods and reagents described herein can be embodied for the improvement of conventional lateral flow immunoassay (LFIA) through increase in sensitivity, reduction in non-specific interference, and ability to quantitate.

The methods and reagents described herein can be embodied for improving upon the state-of-the-art solution phase homogenous assay by using multimeric linkers that can create multivalent VHH binding moieties and enzymatic fragments. Multivalency generally increases binding affinity and avidity by allowing binding to multiple sites on a target, amplifying a single-bond interaction into a stronger overall multi-bond interaction. This creates a higher effective concentration between the interacting molecules, leading to stronger, more specific, and more stable interactions than a single-site (monovalent) interaction could achieve alone.

As used herein, the term “target molecule” refers to a molecule of interest. The target molecule may be a protein, peptide, aptamer, DNA, RNA, PNA, small molecule, or a combination thereof. The target molecule has features that may be superficial (i.e., on the surface) that can be recognized by other binding molecules. The target molecule may have features that are buried. The target molecule may be found in a mixture of other molecules (e.g., a sample from a subject, or an extract). The target molecule preferably has at least two features, either exposed on the surface or buried, that are not the same. The target molecule may be also immunoglobulins present in human serum after a virus infection such as SARS-CoV-2 antibodies, which indicate if the body has an immune response to the SARS-CoV-2 virus either from a past infection or a vaccination. In embodiments, the target molecule may also be a viral particle that is composed of target molecules such as GLRaV-3.

The term “affinity,” as used herein, refers to the degree to which a ligand (as defined further herein) binds to a target protein so as to shift the equilibrium of target protein and ligand toward the presence of a complex formed by their binding. Thus, for example, where a target molecule and a ligand (such as a first molecule having, e.g., a VHH/VNAR, an aptamer, etc.) are combined in relatively equal concentration, a ligand of high affinity will bind to the available target molecule so as to shift the equilibrium toward high concentration of the resulting complex. The dissociation constant is commonly used to describe the affinity between a ligand and a target protein. Typically, the dissociation constant is lower than 10⁻⁵ M. In some embodiments, the dissociation constant is lower than 10⁻⁶ M, and in certain embodiments, the dissociation constant is lower than 10⁻⁷ M. Within the scope of the disclosure, the ligand may be a binding agent, preferably an immunoglobulin, such as an antibody, or an immunoglobulin fragment, such as a VHH or Nanobody, that binds a conformational epitope on a target molecule. It will be appreciated that within the scope of the disclosure, the term “affinity” is used in the context of a binding agent, in particular an immunoglobulin or an immunoglobulin fragment, such as a VHH or Nanobody, that binds a conformational epitope of a target molecule as well as in the context of a test compound (as defined further herein) that binds to a target molecule, more particularly to an orthosteric or allosteric site of a target molecule.

The term “specificity,” as used herein, refers to the ability of a binding agent, in particular an immunoglobulin or an immunoglobulin fragment, such as a VHH or Nanobody, to bind preferentially to one antigen, versus a different antigen, and does not necessarily imply high affinity.

The terms “specifically bind” and “specific binding,” as used herein, generally refers to the ability of a binding agent, in particular, an immunoglobulin, such as an antibody, or an immunoglobulin fragment, such as a VHH or Nanobody, to preferentially bind to a particular antigen that is present in a homogeneous mixture of different antigens. In certain embodiments, a specific binding interaction will discriminate between desirable and undesirable antigens in a sample, in some embodiments more than about 10- to 100-fold or more (e.g., more than about 1,000- or 10,000-fold).

As used herein, a “single-domain antibody” (sdAb) refers to the heavy chain-only antibodies found in camelids and sharks.

As used herein, a “VHH” and a “VNAR” refer to the antigen binding sites of single-domain antibodies derived from camelids and sharks, respectively. VHHs and VNARs may be 20 to 150 amino acids (AA) long. In some embodiments, the VHHs and VNARs comprise about 110 AAs. As used herein, VHH and VNAR are used interchangeably to refer to the antigen binding sites of sdAbs.

As used herein, “about” or “approximately” in reference to a numerical value for a particular parameter is inclusive of the numerical value and a degree of variance from the numerical value that one of ordinary skill in the art would understand is within acceptable tolerances for the particular parameter. For example, “about” or “approximately” in reference to a numerical value may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

As used herein, a “spectral feature” refers to a measurable signal that may be measured spectroscopically by, for example, UV-visible absorption spectroscopy, infrared absorption spectroscopy, fluorescence spectroscopy, luminescence spectroscopy, among other techniques known in the art. The spectral feature may be due to the absorption of light by a molecule or the emission of light by the molecule. In some embodiments, the spectral feature may be measured using a spectrophotometer. In certain embodiments, the spectral feature may be in the visible region, and thus observable by the naked eye.

As used herein, a “protein” refers to a macromolecule made of amino acids (AAs) with N- and C-termini. The protein may be at least 50 AAs, at least 100 AAs, or at least 150 AAs. The protein may be an enzyme widely present in a living organism, which acts as a catalyst to bring about a specific biochemical reaction. The enzyme may be members of the luciferase family found in organisms like fireflies, marine organisms, and fungi that catalyze a reaction with a substrate with or without and oxygen to produce bioluminescence or “cold light.”

As used herein, a “binding moiety” refers to any molecule (large or small) that has affinity and specificity towards the target molecule. The binding moiety may be a macromolecule made of amino acids, i.e., a protein. The binding moiety may be immunoglobulins (IgG) or fragments thereof. The binding moiety may be from the variable domain of the heavy chain of heavy-chain-only antibodies such as those derived from the camelid family (i.e., VHHs). The binding moiety may also be a small molecule such as a low molecular weight drug.

As used herein, the “fusion protein” refers to a macromolecule made of at least two fragments derived from different proteins. Specifically, the “fusion protein” may comprise a binding moiety, linker(s), spacer(s), and fragments of the signal moiety. More specifically, the “first fusion protein” may comprise a first binding moiety, a first linker, spacer(s) and a first fragment of the signal moiety (e.g., N-terminal fragment). The “second fusion protein” may comprise a second binding moiety, a second linker or the same as the first linker or no linker, spacer(s) and a second fragment of the signal moiety (e.g., C-terminal fragment).

As used herein, the “linker” refers to a polypeptide chain that spontaneously forms dimer, trimer, tetramer, or oligomer (e.g., streptavidin, single-stranded DNA binding protein SSB). The incorporation of the linker with this kind of property is one innovative part of this invention that provides significant improvement upon the art.

As used herein, the “spacer” refers to a short flexible peptide sequence of the form (GGGSS)n (SEQ ID NO:2) or its variations (GGSSS)n (SEQ ID NO:3) or (GGGGS)n (SEQ ID NO:4), where n is an integer within a range of from 1 to 10, which provides flexibility, hydrophilicity and minimal interference when joining different protein domains to make fusion proteins. The use of such spacers is a common practice in protein engineering and well known in the art.

As used herein, “nano luciferase” is a generic term referring to any luciferase enzyme derived from the deep-sea shrimp Oplophorus gracilirostris, whether it is molecularly engineered or not.

As used herein, “the small fragment of nano luciferase” may refer to a peptide sequence in an enzyme of deep sea O. gracilirostris, it may also refer to a peptide sequence naturally occurring in species other than O. gracilirostris such as bacteria or humans, which may complement with another fragment of nano luciferase to form an active enzyme.

As used herein, “the large fragment of nano luciferase” may refer to a polypeptide sequence in an enzyme of deep sea O. gracilirostris, whether it is molecularly engineered or not.

As used herein, NanoLuc® is a registered trademark of Promega Corporation (Madison, WI) referring to a certain molecularly engineered luciferase enzyme.

Several VHHs were obtained against the capsid protein of GLRaV-3 by first immunizing an alpaca (under approved IACUC protocol) with the GLRaV-3 capsid protein antigen followed by using a series of standard molecular biology tools including phage display library panning, which are well described in the art. After careful selection and screening, three VHHs were chosen for this study, designated as VHH21 (SEQ ID NO:22), VHH51 (SEQ ID NO:23), and VHH111 (SEQ ID NO:24).

A polyclonal antibody against GLRaV-3, for use as the capture antibody in solid phase assay format, was made by immunizing two rabbits (under approved IACUC protocol) with GLRaV-3 capsid protein and followed by a standard polyclonal antibody production protocol.

The split nano-luciferase system was described earlier by Dixon et al. (2015) and Hall et al. (2012). It consists of two complementary subunits: the larger N-terminal fragment of approximately 17.7-kDa and a C-terminal subunit of 11-amino acid peptide of approximately 1.3 kDa optimized from a peptide library screening to provide a low intrinsic affinity (Kd=190 μM). This low affinity allows for minimal self-association and ensures that a luminescent signal is only generated when two interacting proteins bring the two subunits into close proximity.

The amino acid sequences of N-terminal fragments (LgLuc) and C-terminal fragments (SmLuc) of nano-luciferase are listed as follows:

LgLuc: Amino Acid Sequence (159 amino acids):
(SEQ ID NO: 5)
MVFTLEDFVGDWEQTAAYNLDQVLEQGGVSSLLQNLAVSVTPIQRIVRSG
ENALKIDIHVIIPYEGLSADQMAQIEEVFKVVYPVDDHHFKVILPYGTLV
IDGVTPNMLNYFGRPYEGIAVFDGKKITVTGTLWNGNKIIDERLITPDGS
MLFRVTINS
SmLuc Amino Acid Sequence (11 amino acids):
(SEQ ID NO: 6)
VTGYRLFEEIL

Huang et al. (2023) studied the effect of multimeric nanobody to vascular endothelial growth factor (VEGF) on binding affinity and found that tetravalency increased binding by a factor 134-fold. The multimeric nanobody described in their work was, however, made by tandem linkage of 2, 3, or 4 VHH unit sequentially. Unlike the approach described here, their approach increases the molecular size making it difficult to express. More importantly, it can create potential steric hindrance for recognition and binding, therefore limiting the potential for binding affinity increases. Harnessing the high-affinity, spontaneous tetramerization of proteins such as streptavidin, our method is designed to create VHH multimers with an apparent binding affinity substantially higher than the 134-fold enhancement previously observed.

It was reasoned that if the intrinsic binding affinity between the large and small fragments of nanoluciferase is 190 uM (Kd), their apparent binding affinity (i.e., avidity) will likely increase significantly when the fragments are made tetravalent. VHHs, like any monoclonal antibodies, have a typical affinity Kd constant of nanomolar or sub nanomolar range. The working concentrations of VHH are typically in the range of nanomolar range. Since the nanoluciferase fragments are fused to VHH nanobodies, their working concentrations are also in the nano- to sub nano-molar range under normal assay conditions. Assuming a modest increase of 134-fold increase, the self-association affinity will be approximately Kd=1.5 uM. The fragments will not self-associate to form the active enzyme when present in the nano- to subnano-molar concentration range until they are brought to proximity by way of VHH binding to the target. Once they are brought to proximity, these fragments will self-associate more readily because they have much higher association affinity than the monomeric state. Additionally, tetravalent VHH also increases the binding affinity of VHH to its target, resulting in a significant increase in overall assay sensitivity.

The invention is further described with the use of the following illustrative Examples.

### Example 1

### Key Components of Reagents

A series of twelve (12) protein fusion protein molecules were made according to the concepts depicted in FIG. 1 through FIG. 6 for use in following examples for detecting GLRaV-3. The genes corresponding to the twelve proteins were cloned in an expression vector such as the Pet 24a and expressed in E. coli such strain BL21(DE3) using standard molecular biology tools and conditions. In all of these sequences, the OmpA leader peptide is introduced at the N-terminal end with 21 amino acids, starting with Met-Lys-Lys-Thr-Ala-Ile-Ala-Ile-Ala-Val-Ala-Leu-Ala-Gly-Phe-Ala-Thr-Val-Ala-Gln-Ala- (SEQ ID NO:7). It targets the fusion protein for export across the cytoplasmic membrane to periplasmic space via the sec pathway.

This method utilizes the inherent permeability of a host cell's outer membrane, which allows for the leakage of expressed fusion proteins from the periplasmic space into the growth medium. This advantage enables the cell culture supernatant to serve as a crude source of the fusion protein, thereby simplifying downstream screening procedures and eliminating the time and cost associated with conventional protein purification processes.

Fusion 1
(SEQ ID NO: 8)
VHH21-GGGSSGGGGSGGGGSG-SmLuc-GGGSSGGG-
Streptavidin-HHHHHH
Fusion 2
(SEQ ID NO: 9)
VHH51-GGGSSGGGGSGGGGSG-SmLuc-GGGSSGGG-
Streptavidin-HHHHHH
Fusion 3
(SEQ ID NO: 10)
VHH111-GGGSSGGGGSGGSGG-SmLuc-GGGSSGGG-
Streptavidin-HHHHHH
Fusion 4
(SEQ ID NO: 11)
VHH21-GGGSSGGGGSGGSGG-LgLuc-GGGSSGGG-
Streptavidin-HHHHHH
Fusion 5
(SEQ ID NO: 12)
VHH51-GGGSSGGGGSGGSGG-LgLuc-GGGSSGGG-
Streptavidin-HHHHHH
Fusion 6
(SEQ ID NO: 13)
VHH111-GGGSSGGGGSGGSGG-LgLuc-GGGSSGGG-
Streptavidin-HHHHHH
Fusion 7
(SEQ ID NO: 14)
VHH21-GGGSSGGGGSGGGGSG-SmLuc-HHHHHH
Fusion 8
(SEQ ID NO: 15)
VHH51-GGGSSGGGGSGGGGSG-SmLuc-HHHHHH
Fusion 9
(SEQ ID NO: 16)
VHH111-GGGSSGGGGSGGGGSG-SmLuc-HHHHHH
Fusion 10
(SEQ ID NO: 17)
VHH21-GGGSSGGGGSGGSGG-LgLuc-HHHHHH
Fusion 11
(SEQ ID NO: 18)
VHH51-GGGSSGGGGSGGSGG-LgLuc-HHHHHH
Fusion 12
(SEQ ID NO: 19)
VHH111-GGGSSGGGGSGGSGG-LgLuc-HHHHHH

The first six fusion proteins (Fusion 1 to Fusion 6) contain Streptavidin as the linker in the C-terminal end. The “linker” as defined in this invention does not necessarily mean it has to be situated in-between a VHH and a nanoluciferase fragment. In this case, the linker is situated in the C-terminal end of the fusion protein in the order of VHH-luc fragment-streptavidin. Streptavidin is a 60 kDa bacterial protein from Streptomyces avidinii that is notable for its extremely strong, non-covalent bond with the vitamin biotin and has the following amino acid sequence:

(SEQ ID NO: 20)
MRKIVVAAIAVSLTTVSITASASADPSKDSKAQVSAAEAGITGTWYNQL
GSTFIVTAGADGALTGTYESAVGNAESRYVLTGRYDSAPATDGSGTALG
WTVAWKNNYRNAHSATTWSGQYVGGAEARINTQWLLTSGTTEANAWKST
LVGHDTFTKVKPSAASIDAAKKAGVNNGNPLDAVQQ.

This highly stable interaction, with a dissociation constant (Kd) of 10⁻¹⁴ to 10⁻¹⁵ M, is one of the strongest non-covalent interactions found in nature. This property results in part from its homotetrameric structure composed of four identical protein subunits. Each subunit has an independent but cooperative binding site and each streptavidin molecule can bind up to four biotin molecules.

In this embodiment, the OmpA signal peptide directs the nascent fusion protein into the Sec secretion pathway in the inner membrane of the E. coli host cell. Upon translocation into the periplasmic space, the polypeptide spontaneously refolds and assembles into a tetrameric tertiary structure by virtue of the inherent tetramerization property of the streptavidin moiety.

This method provides a tetravalent VHH and a tetravalent nanoluciferase fragment without increasing the length of the primary amino acid sequence of the fusion protein. Conventional production of multimeric VHHs often involves the use of tandem repeats, which results in a fusion protein of increased size and complexity. The present invention addresses this limitation by providing a process that enables the expression of a minimally sized primary sequence, which nonetheless yields multimeric VHHs. This method circumvents the need for tandem repeats and their associated challenges such as increased molecule size and reduced expression efficiency.

This embodiment describes a method for producing a tetravalent fusion protein, the method comprising of expressing a fusion protein comprising a streptavidin moiety, a variable domain of a heavy chain of a heavy-chain antibody (VHH) moiety, and a nanoluciferase fragment moiety; and directing the fusion protein to the periplasmic space of an E. coli host cell via an OmpA signal peptide; and rapid screening the fusion protein for its designed function by using the cell culture supernatant as a crude source of the fusion protein, thereby and eliminating the time and cost associated with conventional protein purification processes.

In this embodiment, the large nano luciferase fragment LgLuc may be SEQ ID NO:5. It may also be part of the catalytic component of Oplophorus luciferase in the deep-sea shrimp reported by Inouye, S. and Sasaki, S. (2007), the amino acid sequence of which is listed below (196 amino acids):

(SEQ ID NO: 33)
MAYSTLFIIALTAVVTQASSTQKSNLTFTLADFVGDWQQTAGYNQDQVL
EQGGLSSLFQALGVSVTPIQKVVLSGENGLKADIHVIIPYEGLSGFQMG
LIEMIFKVVYPVDDHHFKIILHYGTLVIDGVTPNMIDYFGRPYPGIAVF
DGKQITVTGTLWNGNKIYDERLINPDGSLLFRVTINGVTGWRLCENILA

In this embodiment, the small nano luciferase fragment (SmLuc) may be SEQ ID NO:6. It may also be part of a hypothetical protein in Tateyamaria sp.—a genus of alpha-Proteobacteria belonging to the family Rhodobacteraceae (NCBI Protein Database 2023). The amino acid sequence of the hypothetical protein is listed below (274 amino acids):

(SEQ ID NO: 34)
MNEMLRNQPVLGMFNAYMIRNDKSGAAMLGFLTNTKVTGYKLFEEFLSK
SAKTPIALDGKQQKAVDDVMRGPQKDAAAGLNKLVPGLKKACLTYLDKK
AIPAFYKTKDKPGSVFYQHCRPHAEKNCEGRFGKIAVATKRLGLTDQML
VKEIMVQLYMGNNKSAAAAATKAAKKAGLKMPAVVIVDAVERQRGLIGY
HDVKIDAKSLVFCGFQNVNDKDILKLMKQMVEHHYDKKDSKAKKAFEQI
KKLEPKSSPITKLKYDAFIKLLKKKGTITT

In this embodiment, the small nano luciferase fragment (SmLuc) may come, in particular, from No. 37-47 of the sequence of the hypothetical protein. The amino acid sequence is listed below (11 amino acids):

(SEQ ID NO: 35)
VTGYKLFEEFL

In this embodiment, the small nano luciferase fragment (SmLuc) may be SEQ ID NO:6. It may also be part of a human fibronectin type III domain-containing protein (Kornblihtt, A. R., et. al. 1985). The amino acid sequence of the protein is listed below (421 amino acids):

(SEQ ID NO: 36)
MKKLYLLMLVIGLVLVNACGSDDNGNGTDSESPSAPLNLIASNVSDTSL
ELSWTASTDNTAVTGYRLYEEISGNVTPLGGSTTTYMVTGLTPSTPYKF
YVTAIDAAGNESSQSNTVEISTDEAPLEFLTNLSEMGIFTGDLVNLEPA
ENVQLYELNSTLFTDYAAKQRLIRFPEGQAMRYNNSDQFPVFPDNTLMA
KTFFYYINDQDPGQGKQIIETRLLLKIEGAWQVGNYVWNASQTEATYRE
TGSEIPISYIDGNGDTQNVDYQIPSKADCIICHSNSNTIIPIGPKLRTM
NFVPSYTNMNQLEYFKANGLLEGLGSASSISVLPDWINDVLYTLEERAR
GYIDINCAHCHQPGGAVTNFNIDFQYETPYADTGIYPNRGEIEMRIQST
LPSYRMPQLGRTVVHEEAVAVLIAYLDTL

In this embodiment, the small nano luciferase fragment (SmLuc) may come, in particular, from No. 62-72 of the sequence of the protein. The amino acid sequence is listed below (11 amino acids):

(SEQ ID NO: 37)
VTGYRL YEEIS

Protein fusion molecules of the large fragments and the small fragment with VHHs (e.g., VHH21, VHH51, VHH111) and any of their combinations thereof are part of the methods and reagents disclosed herein and therefore within the scope of this disclosure.

### Example 2

### ELISA Assays for GLRaV-3 Using Fusion Proteins without a Streptavidin Linker

This embodiment aims to demonstrate the concept of the solid phase ELISA for detecting GLRaV-3 as a target molecule employing reagents and methods depicted in FIGS. 1-7 and described in Example 1 using VHH nano-luciferase fragment fusions without a tetrameric linker.

This embodiment particularly aims to demonstrate the concept of the solid phase ELISA for detecting GLRaV-3 capsid protein and GLRaV-3 virus employing reagents and methods depicted in FIGS. 1-7 and described in Example 1 using GLRaV-3-specific VHH nano-luciferase fragment fusions without a tetrameric linker.

A rabbit polyclonal antibody raised against grapevine leafroll associated virus 3 (GLRaV-3) was immobilized on the bottom of 96-well plate for overnight. Grapevine phloem tissues samples both positive and negative of (GRLaV-3) were applied onto the wells and incubated for 90 minutes at 37° C. and washed. A 1:1 mixture of the supernatant from each of the six E. coli overnight cell cultures of the six fusion proteins (i.e., 3×3 matrix) was then applied onto both positive and negative wells and incubated for 90 minutes at 37° C., followed by washing and application of luciferase substrate (Promega Corp, Madison, WI). Luminescence signal was read on a GlowMax luminometer (Promega Corp.).

The six fusion proteins are abbreviated as follows and their detailed structures are described in Example 1:

$$FUSION PROTEIN 7 = VHH 21 - SmLuc - HHHHHH$$

$$FUSION PROTEIN 8 = VHH 51 - SmLuc - HHHHHH$$

$$FUSION PROTEIN 9 = VHH 111 - SmLuc - HHHHHH$$

$$FUSION PROTEIN 10 = VHH 21 - LgLuc - HHHHHH$$

$$FUSION PROTEIN 11 = VHH 51 - LgLuc - HHHHHH$$

$$FUSION PROTEIN 12 = VHH 111 - LgLuc - HHHHHH$$

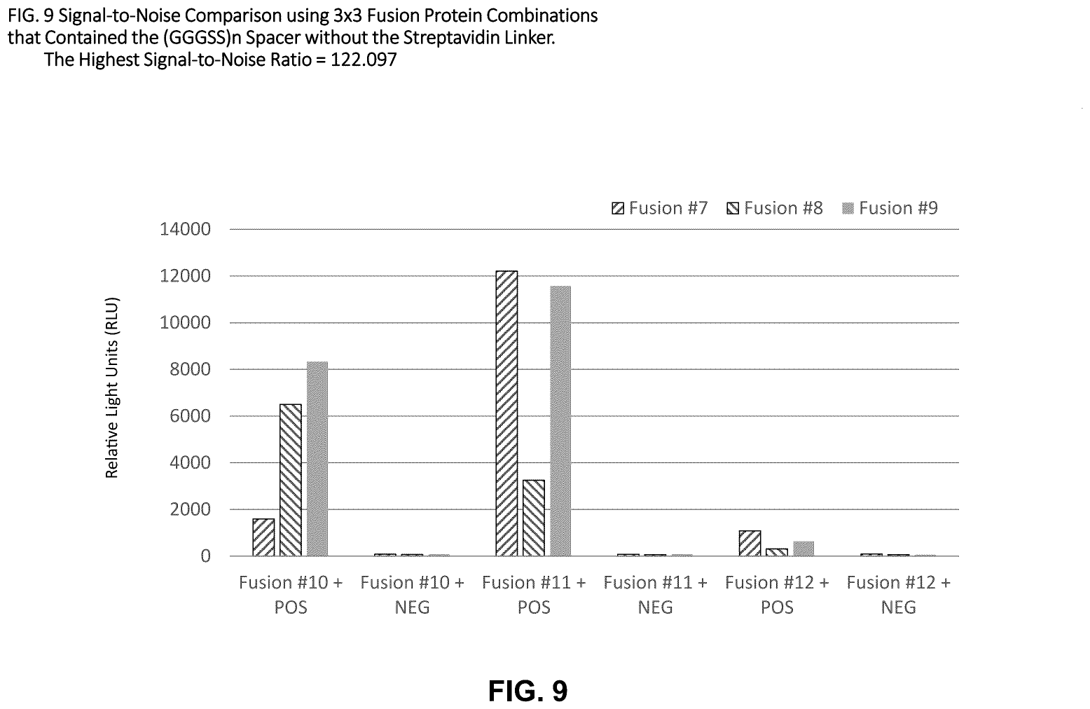

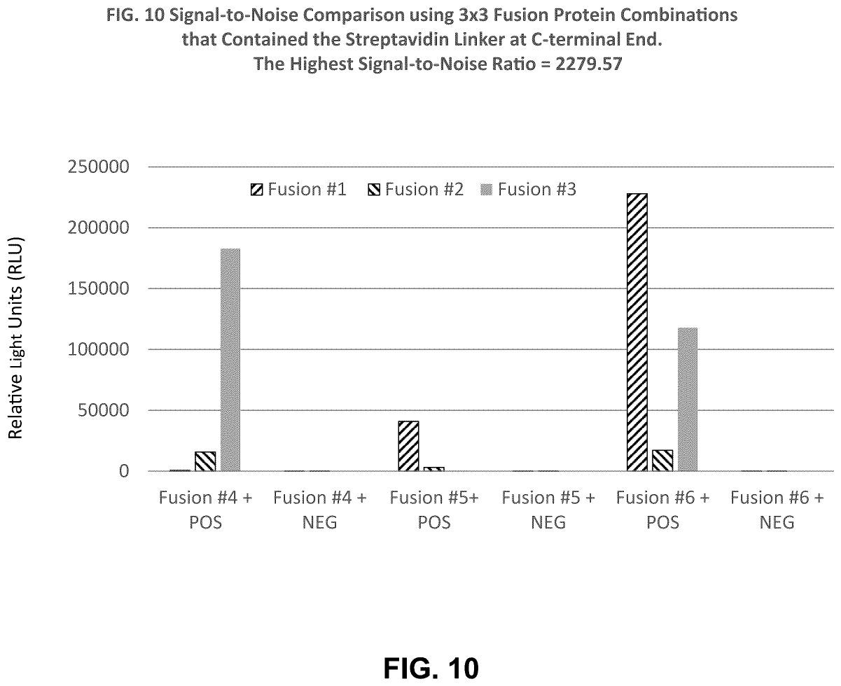

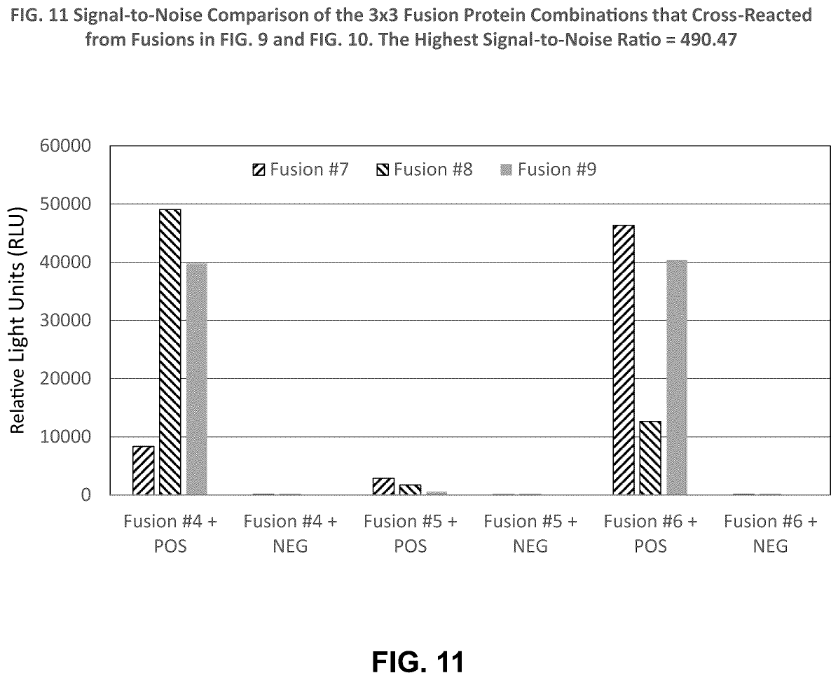

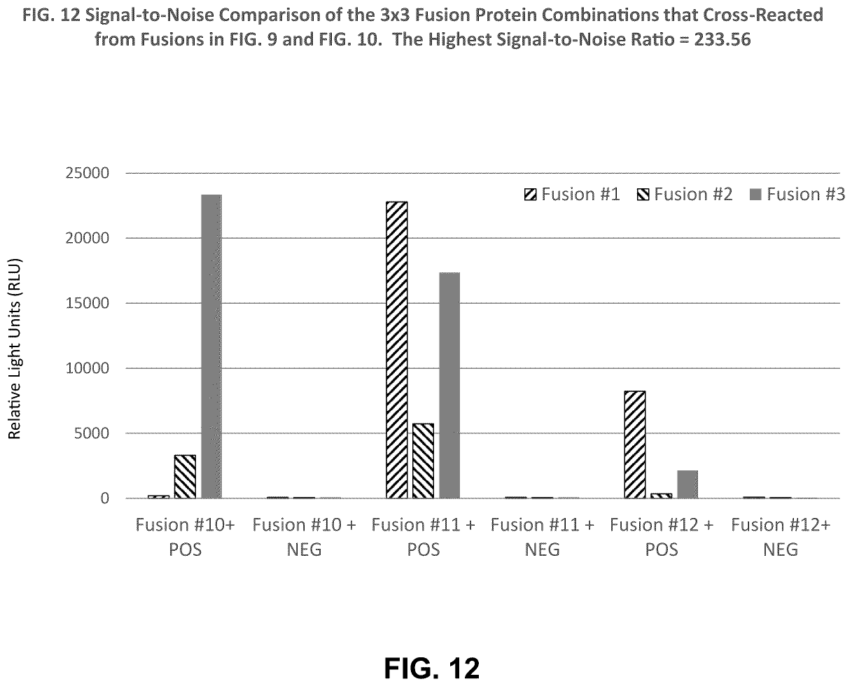

FIG. 9 shows the results of this experiment. The highest signal-to-background noise ratio of SNR=122.09 was observed between Fusion 11 and Fusion 7.

He et al (2023) described two VHHs conjugated to nano-luciferase fragments to develop a sandwich homogenous immunoassay mix-and-read assay for human soluble epoxide hydrolase (HsEH, MW 53 kDa), which has been investigated as a biomarker for various diseases, including different types of cancer, viral hepatitis, and obesity. It reported the highest and best signal-to-noise-ratio of S/N=8.83 and limit of detection of 1.4 ng/ml (LOD). The work of He et al. is the closest example in the published literature to this invention but did not use solid phase assay format nor used any tetrameric linkers.

The method in this example improved upon the art by using a solid phase assay format, the signal-to-noise ratio was 122, which represents a 13.8-fold improvement over the art.

This example describes a new ELISA method where the detection antibody, instead of being labeled with an active enzyme such as alkaline phosphatase as in conventional ELISA, is labeled with enzyme fragments such as N- and C-terminal fragments of nano-luciferase so that the enzyme label is not active until bound to the target molecule resulting an enhancement of signal-to-noise ratio by a factor of at least 13.8-fold relative to comparable data in the art. (He et al. 2023.)

This example additionally describes a new solid phase ELISA method where the detection antibody is comprised of two VHH antibodies, as opposed to one, each fused with the N- and C-terminal fragments of nano-luciferase, respectively. The use of two detection VHHs improves upon the art assay by reducing non-specific interference and increasing assay specificity.

### Example 3

### ELISA Assays for GLRaV-3 Using Fusion Proteins Containing Streptavidin as Linker

This embodiment aims to demonstrate the concept of the solid phase ELISA for detecting a target molecule employing reagents and methods depicted in FIGS. 1-7 and described in Example 1 using VHH nano-luciferase fragment fusions with a tetrameric linker.

This embodiment particularly aims to demonstrate the concept of the solid phase ELISA for detecting GLRaV-3 employing reagents and methods depicted in FIGS. 1-7 and described in Example 1 using VHH nano-luciferase fragment fusions with a tetrameric linker.

A rabbit polyclonal antibody raised against grapevine leafroll associated virus 3 (GLRaV-3) was immobilized on the bottom of 96-well plate for overnight. Grapevine phloem tissues samples both positive and negative for GRLaV-3 were applied onto the wells and incubated for 90 minutes at 37° C. and washed. A 1:1 mixture of the supernatant from each of the six E. coli overnight cell cultures of the six fusion proteins (i.e., 3×3 matrix) were applied onto both positive and negative wells and incubated for 90 minutes at 37° C., followed by washing and application of luciferase substrate (Promega Corp.) Luminescence signal was read on a GlowMax luminometer (Promega Corp.).

The six fusion proteins are abbreviated as follows; their detailed structures are described in Example 1:

$$FUSION PROTEIN 1 = VHH 21 - SmLuc - streptavidin - HHHHHH$$

$$FUSION PROTEIN 2 = VHH 51 - SmLuc - streptavidin - HHHHHH$$

$$FUSION PROTEIN 3 = VHH 111 - SmLuc - streptavidin - HHHHHH$$

$$FUSION PROTEIN 4 = VHH 21 - LgLuc - streptavidin - HHHHHH$$

$$FUSION PROTEIN 5 = VHH 51 - LgLuc - streptavidin - HHHHHH$$

$$FUSION PROTEIN 6 = VHH 111 - LgLuc - streptavidin - HHHHHH$$

FIG. 10 shows the results of this experiment. The highest signal to background noise ratio was SNR=2279.57 between Fusion 6 and Fusion 1.

This experiment was conducted side-by-side in parallel with that described in Example 2 using the exact same sample under the same experimental conditions. The only difference is that the detection fusion proteins contained streptavidin in the C-terminal end of the fusion proteins between VHH and nano-luciferase fragments.

Conventional ELISAs generally have a signal-to-noise ratio of 20-50, depending on assay design and choice of antibodies, labels and substrates. A signal-to-noise ratio of 2279 is unprecedented in the literature, attributable to the little background of enzyme fragments before binding to target, high affinity binding of the tetravalent VHH, and high enzymatic efficiency of tetrameric nano-luciferase after being reassembled upon binding to the target.

This example describes a new ELISA method where the detection antibody, instead of being labeled with an active enzyme such as alkaline phosphatase as in conventional ELISA, is labeled with enzyme fragments such as N- and C-terminal fragments of nano-luciferase so that the enzyme label is not active until bound to the target molecule resulting an enhancement of signal-to-noise ratio by a factor of at least 258-fold relative to comparable data in the art (He et al. 2023) and a factor of at least 19-fold relative to data in Example 2 where no tetrameric linker was used.

This example additionally describes a new solid phase ELISA method where the detection antibody is comprised of two VHH antibodies, as opposed to one in conventional ELISA, each fused with the N- and C-terminal fragments of nano-luciferase, respectively. The use of two detection VHHs improves upon state-the art assay by reducing non-specific interference and increasing assay specificity.

This example further describes a new solid phase ELISA method where the detection moiety is comprised of not only VHH fusion with nano-luciferase fragments but also contains a polypeptide linker (e.g., streptavidin) in the C-terminal end that forms tetramers spontaneously. The tetramerization of streptavidin makes both VHH and nano-luciferase fragments tetravalent and can substantially improve binding affinity of VHH and enzymatic efficient of nano-luciferase due to binding cooperativity and increase in effective local concentration of binding sites.

Although the tetrameric polypeptide linker (e.g., streptavidin) is situated in the C-terminal end of the fusion proteins in this example, its location may be at the C-terminal end or in-between a VHH and a luciferase fragment. The exact location of streptavidin may be assay specific depending on the target and the choice of VHHs and to be experimentally determined for optimal performance.

The tetrameric polypeptide linker may be from the family of single-stranded DNA binding proteins (SSB) including those from thermophilic bacteria such as Thermus aquatic SSB (i.e., TaqSSB).

The polypeptide linker may be dimeric, trimeric, tetrameric and multimeric.

### Example 4

### ELISA Assays for GLRaV-3 Using VHH Fusions with N-Terminal Fragment of Nano-Luciferase with a Steptavidin Linker and that with C-Terminal Fragment without a Steptavidin Linker

This embodiment is aimed to demonstrate the concept of the solid phase ELISA for detecting GLRaV-3 as a target molecule employing reagents and methods depicted in FIGS. 1-7 and described in Example 1 using a combination of VHH fusion proteins with nanoluciferase large fragment (SmLuc) fusions with a tetrameric linker and VHH fusion proteins with nanoluciferase small fragment (LgLuc) without a tetrameric linker.

This embodiment particularly aims to demonstrate the concept of the solid phase ELISA for detecting GLRaV-3 employing reagents and methods depicted in FIGS. 1-7 and described in Example 1 using the combination of VHH nano-luciferase fragment fusions with and without a tetrameric linker.

A rabbit polyclonal antibody raised against grapevine leafroll associated virus 3 (GLRaV-3) was immobilized on the bottom of 96-well plate for overnight. Grapevine phloem tissues samples both positive and negative of (GRLaV-3) were applied onto the wells and incubated for 90 minutes at 37° C. and washed. A 1:1 mixture of the supernatant from each of the six E. coli overnight cell cultures of the six fusion proteins (i.e., 3×3 matrix) were applied onto both positive and negative wells and incubated for 90 minutes at 37° C., followed by washing and application of luciferase substrate (Promega Corp.). Luminescence was read on a GlowMax luminometer (Promega Corp.).

The six fusion proteins are abbreviated as follows; their detailed structures are described in Example 1.

$$FUSION PROTEIN 4 = VHH 21 - LgLuc - streptavidon - HHHHHH$$

$$FUSION PROTEIN 5 = VHH 51 - LgLuc - streptavidon - HHHHHH$$

$$FUSION PROTEIN 6 = VHH 111 - LgLuc - streptavidon - HHHHHH$$

$$FUSION PROTEIN 7 = VHH 21 - SmLuc - HHHHHH$$

$$FUSION PROTEIN 8 = VHH 51 - SmLuc - HHHHHH$$

$$FUSION PROTEIN 9 = VHH 111 - SmLuc - HHHHHH$$

FIG. 11 shows the results of this experiment. The highest signal to background noise ratio was SNR=490.47 between Fusion 4 and Fusion 8.

This experiment was conducted side-by-side in parallel with that described in Example 2 using the exact same sample under the same experimental conditions. The difference is that VHH fusions with nano-luciferase large fragments contain streptavidin in the C-terminal end and that VHH fusions with nano-luciferase small fragments do not contain streptavidin in the C-terminal end.

Conventional ELISAs generally have a signal-to-noise ratio of 20-50, depending on assay design and choice of antibodies, labels and substrates. A signal-to-noise ratio of 490 is unprecedented in the literature, attributable to the little background of enzyme fragments before binding to target, high affinity binding of the tetravalent VHHs in fusions with the large nanoluciferase fragment, and high enzymatic efficiency of tetrameric nano-luciferase after being reassembled upon binding to the target.

This example describes a new ELISA method where the detection antibody, instead of being labeled with an active enzyme such as alkaline phosphatase as in conventional ELISA, is labeled with enzyme fragments such as N- and C-terminal fragments of nano-luciferase so that the enzyme label is not active until bound to the target molecule resulting an enhancement of signal-to-noise ratio by a factor of at least 55-fold relative to comparable data in the art (He et al., 2023) and a factor of 4 relative to data in Example 2.

This example additionally describes a new solid phase ELISA method where the detection antibody is comprised of two VHH antibodies, as opposed to one in conventional ELISA, each fused with the N- and C-terminal fragments of nano-luciferase, respectively. The use of two detection VHHs improves upon state-the art assay by reducing non-specific interference and increasing assay specificity.

This example further describes a new solid phase ELISA method where the detection moiety is comprised of VHH fusions with nano-luciferase large fragment with a polypeptide linker (e.g., streptavidin) that forms tetramers spontaneously, and VHH fusions with small fragment without such a linker. The tetramerization of streptavidin makes both VHH and nano-luciferase fragments tetravalent and can substantially improve binding affinity of VHH and enzymatic efficient of nano-luciferase due to binding cooperativity and increase in effective local concentration of binding sites.

Although the tetrameric polypeptide linker (e.g., streptavidin) is situated in the C-terminal end of the fusion proteins in this example, its location may be at the C-terminal end or in-between a VHH and a luciferase fragment. The exact location of streptavidin may be assay-specific depending on the target and the choice of VHHs and to be experimentally determined for optimal performance.

The tetrameric polypeptide linker may be from the family of single-stranded DNA binding proteins (SSB) including those from thermophilic bacteria such as Thermus aquatic SSB (i.e., TaqSSB).

The polypeptide linker may be dimeric, trimeric, tetrameric and multimeric.

### Example 5

### ELISA Assays for GLRaV-3 Using VHH Fusion Proteins with N-Terminal Fragment of Nano-Luciferase without a Streptavidin Linker and that with C-Terminal Fragment with a Streptavidin Linker

This embodiment is aimed to demonstrate the concept of the solid phase ELISA for detecting a target molecule employing reagents and methods depicted in FIGS. 1-7 and described in Example 1 using a combination of VHH fusion proteins with nanoluciferase small fragment (SmLuc) fusions without a tetrameric linker and VHH fusion proteins with nanoluciferase large fragment (LgLuc) fusions with a tetrameric linker.

This embodiment particularly aims to demonstrate the concept of the solid phase ELISA for detecting GLRaV-3 employing reagents and methods depicted in FIGS. 1-7 and described in Example 1 using the combination of VHH nano-luciferase fragment fusions with and without a tetrameric linker.

A rabbit polyclonal antibody raised against grapevine leafroll associated virus 3 (GLRaV-3) was immobilized on the bottom of 96-well plate for overnight. Grapevine phloem tissues samples both positive and negative of (GRLaV-3) were applied onto the wells and incubated for 90 minutes at 37° C. and washed. A 1:1 mixture of the supernatant from each of the six E. coli overnight cell cultures of the six fusion proteins (i.e., 3×3 matrix) were applied onto both positive and negative wells and incubated for 90 minutes at 37° C., followed by washing and application of luciferase substrate (Promega Corp.) Luminescence signal was read on a GlowMax luminometer (Promega Corp.).

The six fusion proteins were abbreviated as follows; their detailed structures are described in Example 1:

$$FUSION PROTEIN 1 = VHH 21 - SmLuc - streptavidon - HHHHHH$$

$$FUSION PROTEIN 2 = VHH 51 - SmLuc - streptavidon - HHHHHH$$

$$FUSION PROTEIN 3 = VHH 111 - SmLuc - streptavidon - HHHHHH$$

$$FUSION PROTEIN 10 = VHH 21 - LgLuc - HHHHHH$$

$$FUSION PROTEIN 11 = VHH 51 - LgLuc - HHHHHH$$

$$FUSION PROTEIN 12 = VHH 111 - LgLuc - HHHHHH$$

FIG. 12 shows the results of this experiment. The highest signal to background noise ratio was SNR=233.56 between Fusion 10 and Fusion 3.

This experiment was conducted side-by-side in parallel with that described in Example 2 using the exact same sample under the same experimental conditions. The difference is that VHH fusions with nano-luciferase small fragments contain streptavidin in the C-terminal end and that VHH fusions with nano-luciferase large fragments do not contain streptavidin in the C-terminal end.

Conventional ELISAs generally have a signal-to-noise ratio of 20-50, depending on assay design and choice of antibodies, labels and substrates. A signal-to-noise ratio of 233.56 is unprecedented in the literature, attributable to the little background of enzyme fragments before binding to target, high affinity binding of the tetravalent VHH, and high enzymatic efficiency of tetrameric nano-luciferase after being reassembled upon binding to the target.

This example describes a new ELISA method where the detection VHH antibody, instead of being labeled with an active enzyme such as alkaline phosphatase as in conventional ELISA, is labeled with enzyme fragments such as N- and C-terminal fragments of nano-luciferase so that the enzyme label is not active until bound to the target molecule resulting an enhancement of signal-to-noise ratio by a factor of at least 26-fold relative to comparable data in the art (He et al. 2023) and a factor of 1.9 relative to data in Example 2.

This example additionally describes a new solid phase ELISA method where the detection antibody is comprised of two VHH antibodies, as opposed to one in conventional ELISA, each fused with the N- and C-terminal fragments of nano-luciferase, respectively. The use of two detection VHHs improves upon state-the art assay by reducing non-specific interference and increasing assay specificity.

This example further describes a new solid phase ELISA method where the detection moiety is comprised of VHH fusions with nano-luciferase large fragment without a polypeptide linker (e.g., streptavidin) and VHH fusions with small fragment with such a linker that forms tetramers spontaneously. The tetramerization of streptavidin makes both VHH and nano-luciferase fragments tetravalent and can substantially improve binding affinity of VHH and enzymatic efficient of nano-luciferase due to binding cooperativity and increase in effective local concentration of binding sites.

Although the tetrameric polypeptide linker (e.g., streptavidin) is situated in the C-terminal end of the fusion proteins in this example, its location may be at the C-terminal end or in-between a VHH and a luciferase fragment. The exact location of streptavidin may be assay-specific depending on the target and the choice of VHHs and to be experimentally determined for optimal performance.

The tetrameric polypeptide linker may be from the family of single-stranded DNA binding proteins (SSB) including those from thermophilic bacteria such as Thermus aquatic SSB (i.e., TaqSSB).

The polypeptide linker may be dimeric, trimeric, tetrameric and multimeric.

### Example 6

### A Homogenous

### One-Step Mix-and-Read Assay

This embodiment aims to demonstrate the method of the homogenous one-step mix-and-read assay for directly detecting a target, as depicted in FIGS. 1 to 5 without the need to capture the target on solid substrate followed by multiple processing steps as required in conventional ELISA.

This embodiment specifically teaches the application of the methods and reagents disclosed in this invention for directly detecting GLRaV-3 in grapevine plant tissues by employing the best combination of VHH proteins that showed highest signal-to-noise ratio among all combinations studied in Examples 2 through 5. As demonstrated in Example 3 and FIG. 10, the combination of Fusion 6 and Fusion 1 showed highest signal-to-noise ratio of 2279.57. Both contain streptavidin as the tetrameric linker.

$$FUSION PROTEIN 1 = VHH 21 - SmLuc - streptavidin - HHHHHH$$

$$FUSION PROTEIN 6 = VHH 111 - LgLuc - streptavidin - HHHHHH$$

For purpose of this example, ten phloem tissue chips from ten different grapevine cuttings were first added to ten different wells on a 96-well plate. Each phloem chip (ca. 5×5 mm) is taken using a sterile blade from under the lignified tissue and added directly to the well without touching anything besides the blade. Each grapevine cutting was taken from a different vine and prior RT-PCR testing confirmed that out of the ten cuttings, only one was infected with the GLRaV-3 virus and nine were free of infection.

A 1:1 mixture of the supernatant from each E. coli overnight cell culture of the above two fusion proteins, together with the appropriate amount of nano-luciferase substrate (Promega Corp), was added to the ten (10) wells containing the phloem tissue chips on the 96-well plate. Luminescence signal was read on a GlowMax luminometer (Promega Corp).

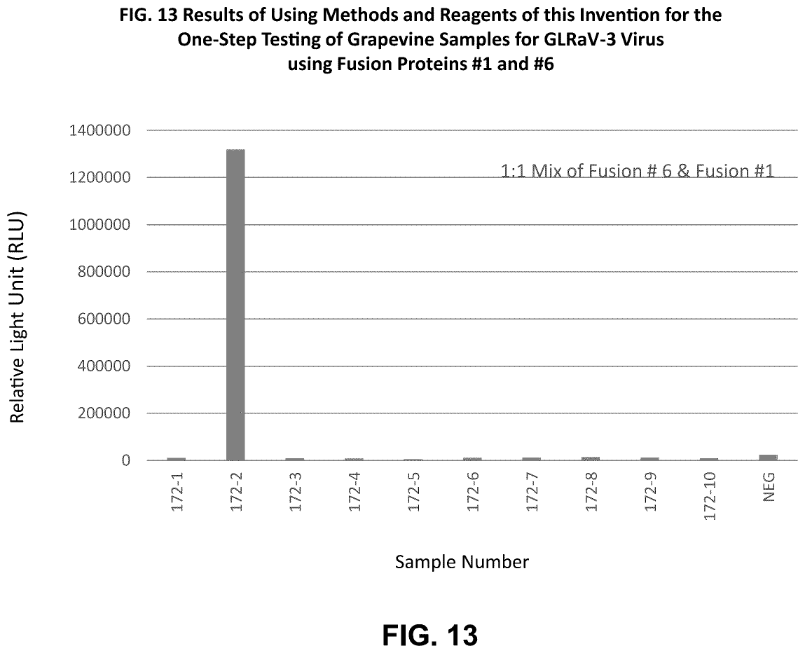

FIG. 13 shows the results of this experiment. Well 2 had a reading of 1,319,140 relative light unit (RLU) and rest nine wells had an average reading of 10,867±5830 (RLU), which is a contrast of 121-fold, indicating cutting 2 was positive for GLRaV-3, consistent with RT-PCR result.

This example teaches a method of using the reagents described in this invention to directly detect a target molecule in crude samples that contain the molecule. The target molecule may be present freely in the sample such as disease biomarkers in the blood or in association of a target in the sample such as the surface antigens of cancer cells, capsid proteins of viruses, membrane proteins of bacteria or outer wall glycoproteins of fungi.

This example specifically teaches a method of using the reagents described in this invention to directly detect the capsid protein molecule of grapevine leaf associated virus type 3 (GLRaV-3) in crude grapevine phloem tissues.

This example more specifically teaches a method of using the reagents described in this invention to directly detect grapevine leaf associated virus type 3 (GLRaV-3) in crude grapevine phloem tissues.

This example discloses a reagent composition comprising of at least two binding moieties such as VHHs or VNARs fused, via a linker, to the N- or C-terminal fragment of a functional protein such as an enzyme or fluorescent protein, respectively, where these fusion proteins are not functionally active until the binding moieties bind to their the target to bring the protein fragments to proximity to restore its functionality (FIG. 1 and FIG. 2).

The fusion protein between the binding moieties (VHHs or VNARs) and the N- or C-terminal fragments of a functional protein may also contain a functional linker that may be positioned in the C-, or N-terminal end, or in-between the two subunits. The linker possesses the property of spontaneously forming tetramers or multimers (e.g., streptavidin or SSB) to make both the binding moieties and the protein fragments multivalent simultaneously. The exact location of the linker may be assay-specific depending on the nature of the target and the choice of VHHs and be experimentally determined for optimal performance.

### Example 7

### Sensitivity Enhancement in Comparison with Conventional ELISA

This embodiment aims to demonstrate the sensitivity enhancement by using methods and reagents disclosed in this invention in Examples 1 through Example 5.

This embodiment specifically teaches the application of the methods and reagents for detecting GLRaV-3 in several serially diluted crude grapevine phloem tissue samples by employing the combination of VHH fusion proteins that showed highest signal-to-noise ratio among all combinations studied in Examples 2 through 5. As shown in Example 3 and FIG. 10, the combination of Fusion 6 and Fusion 1 showed the best and highest signal-to-noise ratio of 2279.57. Both contains streptavidin as the tetrameric linker:

$$FUSION PROTEIN 1 = VHH 21 - SmLuc - streptavidin - HHHHHH$$

$$FUSION PROTEIN 6 = VHH 111 - LgLuc - streptavidin - HHHHHH$$

For purpose of this example, a grapevine phloem tissue sample, known to be positive for GLRaV-3, was homogenized in a sample buffer under standard conditions. The sample is serially diluted from 1× to 3×, 9×, 27× . . . and to 59049× to make eleven (11) serially diluted the samples including the original non-diluted sample. The eleven (11) samples plus a negative control were applied to 12 wells on each of three 96-well plates, respectively. The wells were precoated with a polyclonal capture antibody for the GLRaV-3. The first plate for was processed according to the method described in Example 3 with FUSION 1 and 6 as the detection pair. The second plate was processed according to standard ELISA method using an alkaline phosphatase-labeled detection autobody and para-nitrophenyl phosphate (PNPP) substrate. The third plate was processed the same way except that the substrate was Lumi-Phos 530 (Beckman Coulter Inc.), which is a chemiluminescent substrate commonly used in immunoassays.

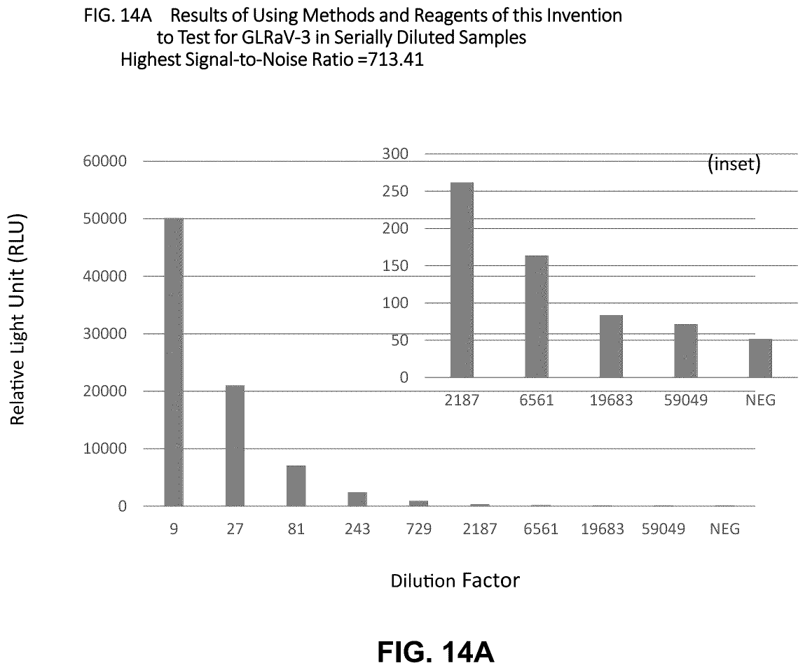

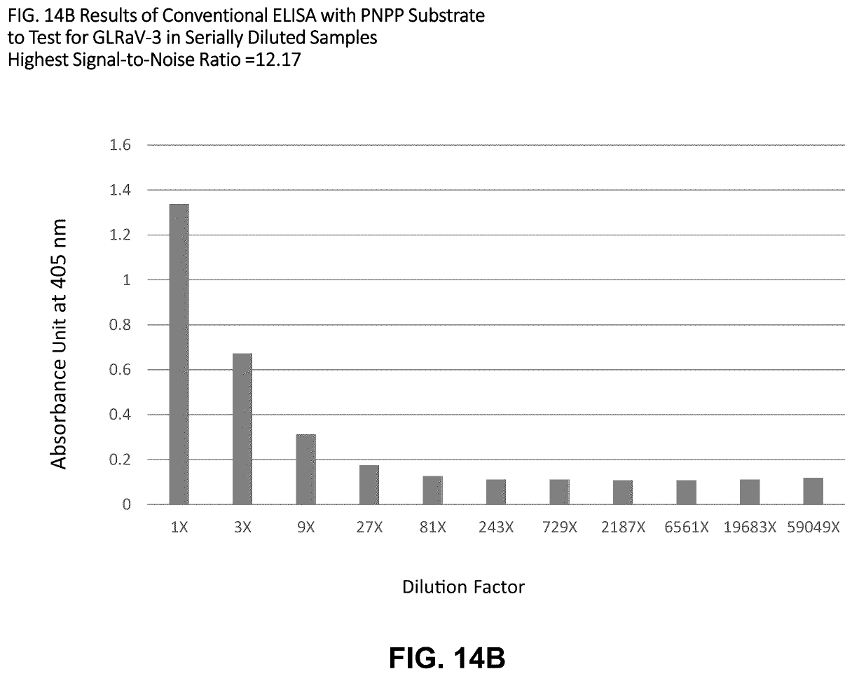

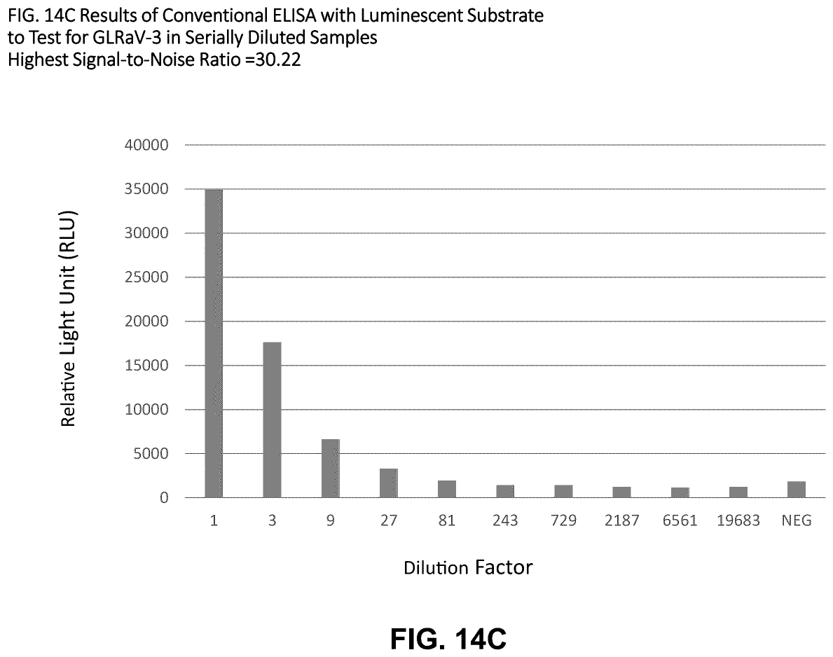

FIG. 14 shows the results of this experiment. Using conventional ELISA with both PNPP and Lumi-Phos 530 substrates, after 27× sample dilution, the signals were indistinguishable from the background noise. By contrast, using the methods and reagents described herein, the signal starts to become indistinguishable after 6561× sample dilution, demonstrating at least a 250-fold enhancement in sensitivity.

No prior art information can be found where a solid phase ELISA using the methods and reagents similar to what described herein provides 250-fold more sensitive than the conventional ELISA for a target molecule.

Ding et al. (2022) compared the assay sensitivity of their method mix-and-read method using VHH fused to nanoluciferase fragments for the detection of 2,4-D herbicide with several other methods. While their method requires fewer steps and saves time, the sensitivity was 3.64 ng/ml (IC50 value). In comparison to sensitivity of other methods that ranged from 1.9 ng/ml to 29.2 ng/ml, the method of Ding et al. did not gain significant improvement in sensitivity.

He et al. (2023) reported a mix-and-read assay for detection of human soluble epoxide hydrolase (HsEH) using two VHHs (VHHA1 (SEQ ID NO:25), VHHA9 (SEQ ID NO:26)) fused to the small and large fragments of nanoluciferase, respectively. The best signal-to-noise ratio was 8.83 and limit of detection was 1.4 ng/ml (LOD). Homogenous assays are generally known to have modest sensitivity. A LOD of 1.4 ng/mL is 28-fold higher than that of 0.05 ng/ml for HsEH using an ELISA method published by the same research group (Li et al., 2018). By contrast, the best signal-to-noise ratio was 2279.57 (Example 3) in this invention and a 250-fold sensitivity improvement was observed in side-by-side experiments for the same target in this Example.

### Example 8

### Sensitivity Enhancement in Comparison with PCR and qPCR

The objective of this Example is the sane as Example 14 except that this example compares the sensitivity levels between that using methods and reagents of this invention and that of qPCR and RT-PCR for detecting a target molecule in general and the capsid protein of GLRaV-3 virus, in particular.

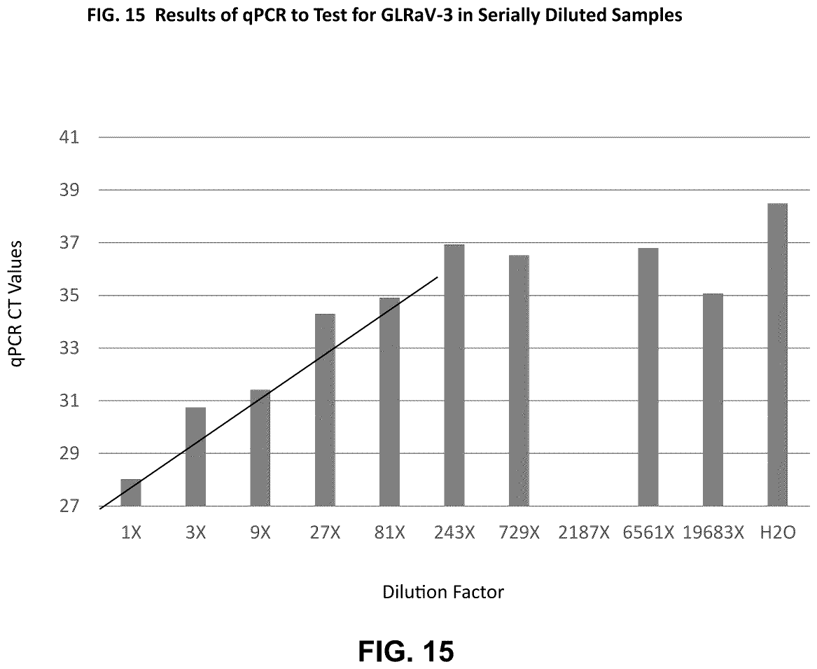

FIG. 15 showing the CT values of qPCR as a function of dilution factor for the same set of serially diluted crude samples as in FIG. 14. The CT values become indistinguishable from the background after 81× dilution. Similar results were obtained when RT-PCR was used to test the same set of serially diluted samples (data not shown). By comparing to data in FIG. 14A, a sensitivity improvement of approximately 81-fold was obtained than RT-PCR and in this experiment.

No prior art information can be found where an ELISA test is 81-fold more sensitive than RT-PCR, qPCR when detecting a virus, bacteria, fungo or any target that contain genetic materials.

### Example 9

### Methods and Reagents for Detecting SARS-CoV-2

A nasopharyngeal swab is obtained from a patient presenting with fever, cough, and shortness of breath. The swab is placed into viral transport medium and aliquoted for testing. The sample is incubated with two fusion proteins prepared according to embodiments of the invention:

The reagents comprise two camelid-derived VHH antibodies (VHH-N4, VHH-N6) specific for a first and second epitopes of the SARS-CoV-2 nucleocapsid (N) protein fused, via a flexible spacer, to the large fragment (LgLuc) of Nano luciferase, and via spacer, with or without a polypeptide linker that possess the property of forming multimers (e.g., tetramers) spontaneously.

Fusion #13
(SEQ ID NO: 38)
VHHN4-GGGSSGGGGSGGGGSG-LgLuc-
GGGSSGGG-Streptavidin-HHHHHH
Fusion #14
(SEQ ID NO: 39)
VHHN6-GGGSSGGGGSGGGGSG-LgLuc-
GGGSSGGG-Streptavidin-HHHHHH
Fusion #15
(SEQ ID NO: 40)
VHHN4-GGGSSGGGGSGGSGG-LgLuc-HHHHHH
Fusion #16
(SEQ ID NO: 41)
VHHN6--GGGSSGGGGSGGSGG-LgLuc-HHHHHH

The reagents further comprise two camelid-derived VHH antibodies (VHH-N4, VHH-N6) specific for a first and second epitopes of the SARS-CoV-2 nucleocapsid (N) protein fused, via a flexible spacer, to the small fragment (SmLuc) of Nano luciferase, and via spacer, with or without a polypeptide linker that possess the property of forming multimers (e.g., tetramers) spontaneously.

Fusion #17
(SEQ ID NO: 42)
VHHN4-GGGSSGGGGSGGGGSG-SmLuc-
GGGSSGGG-Streptavidin-HHHHHH
Fusion #18
(SEQ ID NO: 43)
VHHN6-GGGSSGGGGSGGGGSG-SmLuc-
GGGSSGGG-Streptavidin-HHHHHH
Fusion #19
(SEQ ID NO: 44)
VHHN4-GGGSSGGGGSGGSGG-SmLuc-HHHHHH
Fusion #20
(SEQ ID NO: 45)
VHHN6-GGGSSGGGGSGGSGG-SmLuc-HHHHHH

Using the four small fragment fusions and four large fragment fusions, 4×4=16 combinations are screen against nucleocapsid (N) to obtain the best signal/to noise ratio. The method and process are described in more detail Example 2 through Example #5.

Although depicted to be positioned in the C-terminal end in the sequences hereabove, the linker, for practical purposes, may be positioned in the C-, N-terminal, or in between a VHH and a protein fragment. The exact position of linker is assay specific and has to be determined experimentally for optimal performance.

When SARS-CoV-2 N protein is present in the sample, VHH-N4 and VHH-N6 bind their respective epitopes on the same target molecule. This binding event brings LgLuc and SmLuc into close proximity, reconstituting an active NanoLuc enzyme complex. to generate bioluminescence in the presence of the NanoLuc substrate (furimazine).

The signal is measured using a handheld luminometer or a laboratory instrument. A positive result is indicated by luminescence intensity at least threefold greater than that of a negative control sample (viral transport medium without patient specimen). The limit of detection is anticipated to be at or below 10 μg/mL of N protein, corresponding to clinically relevant viral loads.

The tests may be performed in a mix and read format for rapid sample-to-result such as at point of care or in solid-phase format in a laboratory for additional sensitivity enhancement that is more sensitive than RT-PRC and qPCR.

In alternative embodiments, the same assay format may be configured to detect antibodies against the SARS-CoV-2 spike protein in human serum or plasma samples. In this case, The spike protein is fused to NanoLuc fragments, and binding to the antibodies serves as the bridging event that reconstitutes luciferase activity.

### Example 10

### Incorporation of Thermophile-Derived Split Enzyme Fusion Proteins into a Lateral Flow Assay

A lateral flow assay (LFA) will be retrofitted to incorporate split enzyme fusion proteins of the disclosure for ultrasensitive detection of SARS-CoV-2 nucleocapsid (N) protein.

### Assay Components

Fusion Protein I will comprise a first VHH specific for a first epitope of the SARS-CoV-2 N protein fused, via a (GGGSSGGG)n n spacer (SEQ ID NO:1), to the N-terminal fragment of nanoluciferase. A thermophilic single-stranded DNA binding protein (SSB) derived from Thermus aquaticus will serve as a linker to provide spontaneous tetramerization and stability.

Fusion Protein J will comprise a second VHH specific for a distinct epitope of the N protein fused, via a flexible spacer, to the C-terminal fragment of nanoluciferase. A thermophilic SSB derived from T. thermophilus will likewise serve as a linker.

### Strip Design:

The sample pad will be impregnated with lyophilized Fusion Protein I and Fusion Protein J.

A nitrocellulose membrane will include a test line containing immobilized capture antibodies to SARS-CoV-2 N protein and a control line containing anti-VHH antibodies.

The conjugate pad will include stabilizers such as trehalose and BSA to preserve fusion protein activity under ambient conditions.

### Assay Procedure:

Nasal swab extract from a subject suspected of infection will be applied to the sample pad.

As the extract migrates laterally, the target N protein (if present) will bind Fusion Proteins I and J, bringing the nanoluciferase fragments into close proximity.

The thermophile-derived SSB linkers will multimerize the fusion proteins, facilitating efficient complementation of nanoluciferase into an active enzyme.

The resulting complexes will be captured at the test line by immobilized antibodies.

After binding, the addition of furimazine substrate to the test window will generate a luminescent signal, which will be visible by eye in dim light or read quantitatively with a portable luminometer.

The control line will validate sample migration and reagent integrity.

### Expected Results:

The use of thermophilic linkers will stabilize the fusion proteins under field conditions (20-40° C., variable humidity), reduce false positives by preventing premature enzyme activity, and enhance the signal-to-noise ratio relative to conventional colloidal gold or fluorescent reporter LFAs. The retrofitted LFA is expected to detect SARS-CoV-2 nucleocapsid protein at concentrations as low as 10-100 μg/mL within 15-20 minutes, representing an order of magnitude sensitivity improvement compared to standard LFAs.

### Example 11

### Use of Whole Luciferase in a Multivalent Immunoassay

A diagnostic assay for human prostate-specific antigen (PSA) will be prepared using the disclosure in combination with an intact luciferase reporter.

### Assay Components:

Fusion Protein K will comprise a first VHH specific for a first epitope of PSA fused, via a flexible (GGGSSGGG)n n spacer (SEQ ID NO:1), to an intact luciferase enzyme (e.g., NanoLuc). A thermophilic single-stranded DNA binding protein (SSB) derived from Thermus aquaticus will serve as a multimerizing linker to increase the valency of the fusion.

Fusion Protein L will comprise a second VHH specific for a distinct PSA epitope fused, via a similar flexible spacer, to a second intact luciferase enzyme (e.g., firefly luciferase). A thermophilic SSB derived from T. thermophilus will serve as the linker.

### Assay Workflow:

A microplate will be coated with capture antibodies against PSA.

Patient serum samples will be incubated to allow capture of PSA.

The plate will then be treated with a mixture of Fusion Proteins K and L.

In the presence of PSA, the VHHs of Fusion Proteins K and L will bind to distinct epitopes, creating a sandwich complex.

The intact luciferases carried by the fusion proteins will generate cumulative luminescent signal upon addition of luciferase substrates (e.g., furimazine for NanoLuc and luciferin for firefly luciferase).

### Expected Results:

Unlike split-enzyme complementation assays, where enzyme activity arises only upon fragment complementation, this embodiment leverages intact luciferases carried by both fusion proteins. By employing thermophile-derived multimerizing linkers, the assay provides multivalency, improved avidity, and enhanced retention of fusion proteins on the target. Signal amplification is achieved both through increased binding stability and through the additive activity of multiple intact luciferases bound per target analyte.

### The Retrofitted Assay is Expected to:

Improve sensitivity relative to conventional luciferase-labeled ELISAs by at least 5- to 10-fold due to multivalent presentation of enzyme reporters.

Maintain functional stability under elevated temperatures (30-50° C.) due to the thermophilic SSB linkers.

Allow multiplexing by using distinct intact luciferases with non-overlapping emission spectra (e.g., NanoLuc and firefly luciferase), enabling simultaneous detection of PSA and one or more control analytes in a single well.

### Applications:

This embodiment demonstrates the flexibility of the disclosure in employing not only split enzyme systems but also intact enzymes, offering assay designers the choice between background-free complementation systems and signal-amplified intact enzyme systems depending on the diagnostic context.

### Example 12A

### SARS-CoV-2 Nucleocapsid Detection Using Whole Luciferase

A microplate-based assay will be developed for detecting SARS-CoV-2 nucleocapsid (N) protein.

Fusion Protein M will comprise a VHH specific for a first epitope on the N protein fused, via a (GGGSSGGG)n n spacer (SEQ ID NO:1), to intact NanoLuc luciferase. A thermophilic SSB linker derived from Thermus aquaticus will enable tetramerization and multivalent display.

Fusion Protein N will comprise a second VHH recognizing a different N protein epitope fused to intact firefly luciferase, also tethered via a thermophilic SSB linker (e.g., T. thermophilus).

In the presence of SARS-CoV-2 nucleocapsid protein, both fusion proteins will bind simultaneously, sandwiching the analyte. Signal readout will be achieved by sequential or multiplexed addition of furimazine and luciferin substrates. Dual-luciferase signals will enable higher sensitivity and ratiometric normalization.

### Expected Results:

Sensitivity improved at least 10× compared to standard ELISA.

Multiplexing capability via dual-luciferase emission spectra.

Thermophilic SSB linkers will preserve reagent stability under field conditions.

### Example 12B

### Detection of Grapevine Leafroll-Associated Virus 3 (GLRaV-3) Using Whole Luciferase

A lateral flow strip test will be prepared for detection of GLRaV-3 capsid protein.

Fusion Protein O will comprise a VHH against a first epitope of the GLRaV-3 capsid fused to intact NanoLuc luciferase with a thermophilic SSB (T. aquaticus) as a linker.

Fusion Protein P will comprise a second VHH recognizing a non-overlapping capsid epitope fused to intact Renilla luciferase with a thermophilic SSB (T. thermophilus) linker.

On a lateral flow strip, the viral antigen will capture both Fusion Proteins O and P at the test line. After migration, addition of furimazine and coelenterazine substrates will generate luminescence at distinct wavelengths, enabling dual confirmation.

### Expected Results:

At least an order-of-magnitude sensitivity improvement over conventional colloidal gold LFAs.

Stable reagents for vineyard field testing (ambient 20-40° C.).

Multiplex-ready design for future adaptation to detect multiple grapevine viruses.

### Example 12C

### Detection of Escherichia coli O157:H7 Using Whole Luciferase

A food safety assay will be configured for detection of E. coli O157:H7 in, e.g., ground beef homogenates.

Fusion Protein Q will comprise a VHH against an outer membrane protein of E. coli O157:H7 fused to intact NanoLuc luciferase via a flexible peptide spacer and thermophilic SSB linker.

Fusion Protein R will comprise a second VHH recognizing a distinct OMP epitope fused to intact firefly luciferase with a thermophilic SSB linker.

When present in a sample, the bacterium will be simultaneously bound by Fusion Proteins Q and R. After washing, substrates for NanoLuc and firefly luciferase will be added, and luminescent output will be quantified.

### Expected Results:

Sensitivity to detect ≤10² CFU/mL within 30 minutes.

Signal amplification due to multiple intact luciferases per bacterium.

Robust reagent stability during ambient food-testing conditions.

### Example 13

### Detection of SARS-CoV-2 Nucleocapsid Protein Using Thermophile-Derived Enzyme Linkers

A nasal swab sample is collected from a human subject suspected of SARS-CoV-2 infection. The sample is eluted into a buffered solution containing detergents and reducing agents suitable for viral lysis while preserving protein epitopes.

### Two Fusion Proteins are Prepared:

1. Fusion Protein A will comprise a first VHH specific for a first epitope of the SARS-CoV-2 nucleocapsid (N) protein, joined via a flexible peptide spacer ((GGGSSGGG)n) to the N-terminal fragment of nanoluciferase. A single-stranded DNA binding protein (SSB) derived from Thermus aquaticus will be included as a linker moiety. The SSB will spontaneously form a tetramer when expressed, thereby rendering the VHH-Nluc fusion multivalent.

2. Fusion Protein B will comprise a second VHH specific for a distinct epitope of the SARS-CoV-2 N protein, joined via a flexible peptide spacer to the C-terminal fragment of nanoluciferase. Fusion Protein B will likewise contain a thermophilic SSB linker from T. thermophilus that spontaneously tetramerizes upon recombinant expression.

In the absence of the SARS-CoV-2 nucleocapsid protein, the nanoluciferase fragments will remain separated and non-functional. When the target nucleocapsid protein is present, the two VHHs will bind their respective epitopes, bringing the nanoluciferase fragments into close proximity. Assisted by the tetravalent multimerization properties of the thermophile-derived SSB linkers, the fragments will complex to form active nanoluciferase.

Upon addition of the substrate (furimazine), a luminescent signal will be generated. Signal intensity will correlate with the concentration of the viral nucleocapsid protein in the sample.

### Expected Results

The use of thermophilic SSB linkers will provide enhanced assay sensitivity due to increased stability at elevated temperatures (e.g., 45-60° C.), resistance to denaturation, and improved avidity from multivalent VHH presentation. Compared to assays employing flexible peptide linkers alone, the thermophile-enzyme linked assay is expected to achieve at least a 10-50-fold increase in signal-to-noise ratio, enabling detection of SARS-CoV-2 nucleocapsid protein at sub-picogram per milliliter concentrations in clinical matrices.

### Example 14

### Detection of GLRaV-3 Using Thermophilic Enzyme Linkers

Crude phloem tissue will be collected from grapevine vines suspected of GLRaV-3 infection. Tissue will be macerated in extraction buffer containing 50 mM Tris-HCl (pH 8.0), 150 mM NaCl, and 0.05% Tween-20 to release soluble viral capsid proteins.

### Two Fusion Proteins are Prepared:

1. Fusion Protein C will comprise a first VHH specific for a first epitope of the GLRaV-3 capsid protein. This VHH will be fused, via a flexible (GGGSSGGG)n spacer (SEQ ID NO:1), to the N-terminal fragment of nanoluciferase. A thermophilic single-stranded DNA binding protein (SSB) derived from T. aquaticus will be incorporated as a linker to drive spontaneous tetramerization of the fusion construct.

2. Fusion Protein D will comprise a second VHH recognizing a distinct epitope on the GLRaV-3 capsid protein. This VHH will be fused, via a similar flexible spacer, to the C-terminal fragment of nanoluciferase. A thermophilic SSB linker derived from T. thermophilus will be included to provide multimerization and thermostability.

When Fusion Protein C and Fusion Protein D are incubated with crude grapevine extracts containing GLRaV-3, the specific binding of VHHs to capsid protein epitopes will bring the nanoluciferase fragments into close proximity. The multivalency and heat-stable properties of the thermophile-derived SSB linkers will facilitate efficient complexation of the nanoluciferase fragments to reconstitute a catalytically active enzyme.

After addition of furimazine substrate, a luminescent signal will be detected. The signal intensity will be directly correlated with the presence and concentration of GLRaV-3 capsid proteins in the plant extract.

### Expected Results

The assay employing thermophilic SSB linkers will demonstrate improved stability under vineyard or field-testing conditions (ambient 20-40° C., variable humidity) compared to conventional peptide linkers. Signal-to-noise ratios are expected to exceed those of assays using non-thermophilic linkers by at least one order of magnitude. The method will enable rapid detection of GLRaV-3 in grapevine tissue within 20-30 minutes, supporting early identification and culling of infected vines to prevent spread of the disease.

### Example 15

### Detection of Escherichia coli O157:H7 Using Thermophile-Derived Enzyme Linkers

A food sample (e.g., ground beef homogenate) is prepared by mixing with phosphate-buffered saline containing 0.1% TWEEN®-20 and centrifuged to remove large particulates. The supernatant will be subjected to analysis for the presence of E. coli O157:H7.

### Two Fusion Proteins are Prepared:

1. Fusion Protein E will comprise a first VHH specific for an epitope on the E. coli O157:H7 outer membrane protein (OMP). This VHH will be fused, via a flexible (GGGSSGGG)n spacer (SEQ ID NO:1), to the N-terminal fragment of nanoluciferase. A thermophilic single-stranded DNA binding protein (SSB) derived from T. aquaticus will be incorporated as a linker moiety to enable spontaneous tetramerization and multivalency.

2. Fusion Protein F will comprise a second VHH recognizing a distinct epitope of the E. coli O157:H7 OMP. This VHH will be fused to the C-terminal fragment of nanoluciferase, again with a thermophilic SSB linker derived from T. thermophilus to impart multimerization and thermostability.

In the absence of the target bacterium, the nanoluciferase fragments will remain non-functional. Upon addition of a food extract containing E. coli O157:H7, the VHHs will bind to distinct epitopes on the bacterial surface proteins, bringing the N- and C-terminal fragments of nanoluciferase into close proximity. Assisted by the tetravalent properties of the thermophile-derived SSB linkers, the fragments will complex to restore full enzymatic activity.

Following addition of furimazine substrate, a luminescent signal will be generated. Luminescence intensity will correspond to the bacterial load in the tested sample.

### Expected Results

Use of thermophile-derived SSB linkers will allow the assay to operate effectively under diverse food-processing conditions, including elevated temperatures (30-45° C.) and complex matrices containing fats and proteins. Compared to peptide linker-only constructs, the thermophile-enabled constructs are expected to deliver at least 10-100× improvement in signal-to-noise ratio, allowing rapid detection of E. coli O157:H7 at levels of ≤10² CFU/mL in food extracts within 30 minutes. This performance surpasses conventional ELISA and lateral flow immunoassays, providing a rapid, ultrasensitive, and field-deployable test for bacterial contamination.

### Example 16

### Retrofitting a Conventional ELISA with Thermophile-Linked Split Enzyme Fusion Proteins

A conventional ELISA kit for detection of human prostate-specific antigen (PSA) is retrofitted to employ split enzyme fusion proteins according to the disclosure.

### Baseline Assay:

In a conventional ELISA, capture antibody is immobilized on the bottom of 96-well plates, patient serum is applied, and a single enzyme-labeled detection antibody (e.g., alkaline phosphatase or horseradish peroxidase) generates a colorimetric signal in the presence of substrate. Background activity often arises due to nonspecific binding of the enzyme label, reducing the signal-to-noise ratio and assay sensitivity.

### Retrofitted Assay Using Disclosure Components:

In the retrofitted assay, the detection reagent will no longer be a single enzyme-labeled antibody. Instead, two detection reagents will be used:

1. Fusion Protein G will comprise a first VHH specific for a first epitope on PSA, fused via a flexible (GGGSSGGG)n spacer (SEQ ID NO:1) to the N-terminal fragment of nanoluciferase. A thermophilic single-stranded DNA binding protein (SSB) derived from Thermus aquaticus will serve as a linker to provide multivalency and stability.

2. Fusion Protein H will comprise a second VHH specific for a distinct epitope on PSA, fused to the C-terminal fragment of nanoluciferase, also containing a thermophilic SSB linker derived from T. thermophilus.

### Assay Workflow:

1. A capture antibody against PSA will be coated on ELISA plates.

2. Patient serum samples will be incubated in wells, allowing PSA to bind to the immobilized capture antibody.

3. Instead of a conventional single detection antibody-enzyme conjugate, a mixture of Fusion Protein G and Fusion Protein H will be applied.

4. In the presence of PSA, the two VHHs will bind to distinct epitopes, bringing the nanoluciferase fragments into proximity. The thermophile-derived SSB linkers will tetramerize, enhancing local concentration and driving efficient complementation.

5. After washing, furimazine substrate will be added, and luminescent signal will be measured by a luminometer.

### Expected Results:

The retrofitted ELISA will require fewer washing steps, since the enzyme is only active after both detection VHHs bind PSA, reducing background signal.

Compared to the conventional ELISA signal-to-noise ratio (˜20-50), the retrofitted assay is expected to achieve improvements of at least 10- to 50-fold due to (i) low background from inactive split enzyme fragments, (ii) enhanced avidity from thermophilic SSB multimerization, and (iii) higher catalytic turnover of reconstituted nanoluciferase.

Detection sensitivity is expected to reach sub-picogram per milliliter PSA concentrations, significantly improving upon conventional colorimetric ELISAs.

Because of the thermostability of the thermophilic linkers, the retrofitted kit will maintain performance even when stored or transported without refrigeration.

### Example 17

### Methods and Reagents for Detecting Human Soluble Epoxide Hydrolyase (HsEH)

The reagents comprise two camelid-derived VHH antibodies (VHH-A1, VHH-A9) specific for a first and second epitopes of HsEH) fused, via a flexible spacer, to the large fragment (LgLuc) of Nano luciferase, and via spacer, with or without a polypeptide linker that possess the property of forming multimers (e.g., tetramers) spontaneously.

Fusion #21
(SEQ ID NO: 46)
VHHA1-GGGSSGGGGSGGGGSG-LgLuc-
GGGSSGGG-Streptavidin-HHHHHH
Fusion #22
(SEQ ID NO: 47)
VHHA9-GGGSSGGGGSGGGGSG-LgLuc-
GGGSSGGG-Streptavidin-HHHHHH
Fusion #23
(SEQ ID NO: 48)
VHHA1-GGGSSGGGGSGGSGG-LgLuc-HHHHHH
Fusion #24
(SEQ ID NO: 49)
VHHA9--GGGSSGGGGSGGSGG-LgLuc-HHHHHH

The reagents further comprise two camelid-derived VHH antibodies (VHH-A1, VHH-A9) specific for a first and second epitopes of HsEH fused, via a flexible spacer, to the small fragment (SmLuc) of Nano luciferase, and via spacer, with or without a polypeptide linker that possesses the property of forming multimers (e.g., tetramers) spontaneously.

Fusion #25
(SEQ ID NO: 50)
VHHA1-GGGSSGGGGSGGGGSG-SmLuc-
GGGSSGGG-Streptavidin-HHHHHH
Fusion #26
(SEQ ID NO: 51)
VHHA9-GGGSSGGGGSGGGGSG-SmLuc-
GGGSSGGG-Streptavidin-HHHHHH
Fusion #27
(SEQ ID NO: 52)
VHHA1-GGGSSGGGGSGGSGG-SmLuc-HHHHHH
Fusion #28
(SEQ ID NO: 53)
VHHA9-GGGSSGGGGSGGSGG-SmLuc-HHHHHH

Using the four small fragment fusions and four large fragment fusions, 4×4=16 combinations are screened against human soluble epoxide hydrolyase protein to obtain the best signal-to-noise ratio. The method and process are described in more detail Example 2 through Example #5.

Although depicted to be positioned in the C-terminal end in the sequences hereabove, the linker, for practical purposes, may be positioned in the C-, N-terminal, or in between a VHH and a protein fragment. The exact position of the linker is assay specific and must be determined experimentally for optimal performance.

When HsEH protein is present in the sample, VHH-A1 and VHH-A9 bind their respective epitopes on the same target molecule. This binding event brings LgLuc and SmLuc into close proximity, reconstituting an active NanoLuc enzyme complex to generate bioluminescence in the presence of the NanoLuc substrate (furimazine).

The signal is measured using a handheld luminometer or a laboratory instrument. A positive result is indicated by luminescence intensity at least threefold greater than the standard deviation of a negative control sample. The limit of detection is anticipated to be at or below 10 μg/mL, corresponding to clinically relevant HsEH levels

The tests may be performed in a mix-and-read format for rapid sample-to-result readout such as at point of care or in solid-phase format in a laboratory for additional sensitivity enhancement.

### Example 18

### Methods and Reagents for Detecting Grapevine Fanleaf Virus (FL)

The reagents comprise two camelid-derived VHH antibodies (VHH-F1, VHH-F7) specific for a first and second epitopes of the grapevine fanleaf capsid protein fused, via a flexible spacer, to the large fragment (LgLuc) of Nano luciferase, and via spacer, with or without a polypeptide linker that possesses the property of forming multimers (e.g., tetramers) spontaneously.

Fusion #29
(SEQ ID NO: 54)
VHHF1-GGGSSGGGGSGGGGSG-LgLuc-
GGGSSGGG-Streptavidin-HHHHHH
Fusion #30
(SEQ ID NO: 55)
VHHF7-GGGSSGGGGSGGGGSG-LgLuc-
GGGSSGGG-Streptavidin-HHHHHH
Fusion #31
(SEQ ID NO: 56)
VHHF1-GGGSSGGGGSGGSGG-LgLuc-HHHHHH
Fusion #32
(SEQ ID NO: 57)
VHHF7--GGGSSGGGGSGGSGG-LgLuc-HHHHHH

The reagents further comprise two camelid-derived VHH antibodies (VHH-F1, VHH-F7) specific for a first and second epitopes of the grapevine fanleaf capsid protein fused, via a flexible spacer, to the small fragment (SmLuc) of Nano luciferase, and via spacer, with or without a polypeptide linker that possess the property of forming multimers (e.g., tetramers) spontaneously.

Fusion #33
(SEQ ID NO: 58)
VHHF1-GGGSSGGGGSGGGGSG-SmLuc-
GGGSSGGG-Streptavidin-HHHHHH
Fusion #34
(SEQ ID NO: 59)
VHHF7-GGGSSGGGGSGGGGSG-SmLuc-
GGGSSGGG-Streptavidin-HHHHHH
Fusion #35
(SEQ ID NO: 60)
VHHF1-GGGSSGGGGSGGSGG-SmLuc-HHHHHH
Fusion #36
(SEQ ID NO: 61)
VHHF7-GGGSSGGGGSGGSGG-SmLuc-HHHHHH

Using the four small fragment fusions and four large fragment fusions, 4×4=16 combinations are screened against grapevine fanleaf positive phloem tissues to obtain the best signal/to noise ratio. The method and process are described in more detail Example 2 through Example #5.

Although depicted to be positioned in the C-terminal end in the sequences hereabove, the linker, for practical purposes, may be positioned in the C-, N-terminal, or in between a VHH and a protein fragment. The exact position of the linker is assay specific and has to be determined experimentally for optimal performance.

When grapevine fanleaf virus is present in the sample, VHH-F1 and VHH-F7 bind their respective epitopes on the same target molecule. This binding event brings LgLuc and SmLuc into close proximity, reconstituting an active NanoLuc enzyme complex to generate bioluminescence in the presence of the NanoLuc substrate (furimazine).

The signal is measured using a handheld luminometer or a laboratory instrument. A positive result is indicated by luminescence intensity at least threefold greater than the standard deviation of that of a negative control sample (viral transport medium without patient specimen). The limit of detection is anticipated to be at least 10-fold more sensitive than the state of art ELISA test.

The tests may be performed in a mix and read format for rapid sample-to-result such as at point of care or in solid-phase format in a laboratory for additional sensitivity enhancement that is more sensitive than RT-PRC and qPCR.

### Example 19

### Methods and Reagents for Detecting Prostate Surface Antigen (PSA)

The reagents comprise two camelid-derived VHH antibodies (VHH-P33, VHH-P53) specific for a first and second epitopes of the prostate surface antigen fused, via a flexible spacer, to the large fragment (LgLuc) of Nano luciferase, and via spacer, with or without a polypeptide linker that possesses the property of forming multimers (e.g., tetramers) spontaneously.

Fusion #37
(SEQ ID NO: 62)
VHHP33-GGGSSGGGGSGGGGSG-LgLuc-
GGGSSGGG-Streptavidin-HHHHHH
Fusion #38
(SEQ ID NO: 63)
VHHP53-GGGSSGGGGSGGGGSG-LgLuc-
GGGSSGGG-Streptavidin-HHHHHH
Fusion #39
(SEQ ID NO: 64)
VHHP33-GGGSSGGGGSGGSGG-LgLuc-HHHHHH
Fusion #40
(SEQ ID NO: 65)
VHHP53--GGGSSGGGGSGGSGG-LgLuc-HHHHHH

The reagents further comprise two camelid-derived VHH antibodies (VHH-P33, VHH-P53) specific for a first and second epitopes of prostate surface antigen protein fused, via a flexible spacer, to the small fragment (SmLuc) of Nano luciferase, and via spacer, with or without a polypeptide linker that possesses the property of forming multimers (e.g., tetramers) spontaneously.

Fusion #41
(SEQ ID NO: 66)
VHHP33-GGGSSGGGGSGGGGSG-SmLuc-
GGGSSGGG-Streptavidin-HHHHHH
Fusion #42
(SEQ ID NO: 67)
VHHP53-GGGSSGGGGSGGGGSG-SmLuc-
GGGSSGGG-Streptavidin-HHHHHH
Fusion #43
(SEQ ID NO: 68)
VHHP33-GGGSSGGGGSGGSGG-SmLuc-HHHHHH
Fusion #44
(SEQ ID NO: 69)
VHHP53-GGGSSGGGGSGGSGG-SmLuc-HHHHHH

Using the four small fragment fusions and four large fragment fusions, 4×4=16 combinations are screened against prostate surface antigen to obtain the best signal-to-noise ratio. The method and process are described in more detail Example 2 through Example #5.

Although depicted to be positioned in the C-terminal end in the sequences hereabove, the linker, for practical purposes, may be positioned in the C-, N-terminal, or in between a VHH and a protein fragment. The exact position of the linker is assay specific and has to be determined experimentally for optimal performance.

When prostate surface antigen target is present in the sample, VHH-P33 and VHH-P53 bind their respective epitopes on the same target molecule. This binding event brings LgLuc and SmLuc into close proximity, reconstituting an active NanoLuc enzyme complex to generate ioluminescence in the presence of the NanoLuc substrate (furimazine).

The signal is measured using a handheld luminometer or a laboratory instrument. A positive result is indicated by luminescence intensity at least threefold greater than the standard deviation of the negative control sample. The limit of detection is anticipated to be at or below 10 μg/mL of prostate surface antigen, corresponding to clinically relevant levels.

The tests may be performed in a mix and read format for rapid sample-to-result such as at point of care or in solid-phase format in a laboratory for additional sensitivity.

In each of fusion protein sequences (Fusion #13-#44) described in Example 9 and 17-19 above, the OmpA leader peptide is introduced at the N-terminal end with 21 amino acids, starting with Met-Lys-Lys-Thr-Ala-Ile-Ala-Ile-Ala-Val-Ala-Leu-Ala-Gly-Phe-Ala-Thr-Val-Ala-Gln-Ala- (SEQ ID NO:7). It targets the fusion protein for export across the cytoplasmic membrane to periplasmic space via the sec pathway where the leader peptide is cleaved off at the Ala-Gin-Ala site.

This method utilizes the inherent permeability of a host cell's outer membrane, which allows for the leakage of expressed fusion proteins from the periplasmic space into the growth medium. This advantage enables the cell culture supernatant to serve as a crude source of the fusion protein for rapid screening and testing, thereby eliminating the laborious and costly protein purification processes.

### REFERENCES

The contents of each reference below are incorporated in its entirety herein by reference.
Y. Ding, J. Li, J. R. Enterina, Y. Shen, I. Zhang, P. H. Tewson, . . . R. E. Campbell (2015). Ratiometric biosensors based on dimerization-dependent fluorescent protein exchange. Nature Methods, 195-198.
K. B. Fuller, J. M. Alston, and D. A. Golino (2013). The benefits from certified virus-free nursery stock: A case study of grapevine leafroll-3 in the North Coast region of California. Robert Mondavi Institute-Center for Wine Economics, 35.
He et al., “A mix-and-read nanobody-based sandwich homogeneous split-luciferase assay for the rapid detection of human soluble epoxide hydrolase,” Anal. Chem. 2023 April 11; 95(14): 6038-6045. doi:10.1021/acs.analchem.3c00079.
K. E. Luker, M. C. Smith, G. D. Luker, S. T. Gammon, H. Piwnica-Works, and D. Piwnica-Worms (2004). Kinetics of regulated protein-protein interactions revealed with firefly luciferase complementation imaging in cells and living animals. PNAS, 12288-12293.
D. R. Maass, J. Sepulveda, A. Pernthaner, and C. B. Shoemaker (2007). Alpaca (Lama pacos) as a convenient source of recombinant camelid heavy chain antibodies (VHHs). Journal of Immunological Methods, 13-25.
H. J. Maree, R. P. Almeida, R. Bester, K. M. Chooi, D. Cohen, V. V. Dolja, . . . J. T. Burger (2013). Grapevine leafroll-associated virus 3. Frontiers in Microbiology, 1-21.
J. Robottom (2018). A Split Enzyme for Biosensors. Leeds: University of Leeds.
R. van der Linden, B. de Geus, W. Stok, W. Bos, D. van Wassenaar, T. Verrips, and L. Frenken (2000). Induction of immune responses and molecular cloning of the heavy chain antibody repertoire of Lama glama. 185-195.
Z. Yao, L. Drecun, F. Aboualizadeh, S. J. Kim, Z, Li, H. Wood, . . . I. Stagljar (2021). A homogeneous split-luciferase assay for rapid and and sensitive detection of anti-SARS-CoV-2 antibodies. Nature Communications, 1-8.
WO 2023/235955 A1 (Dec. 14, 2023) to Yao et al.
A. S. Dixon et al., “NanoLuc Complementation Reporter Optimized for Accurate Measurement of Protein Interactions in Cells,” ACS Chemical Biology, Vol. 11/Issue 2, Nov. 16, 2015.
M. P. Hall et al., “Engineered Luciferase Reporter from a Deep Sea Shrimp Utilizing a Novel Imidazopyrazinone Substrate,” ACS Chemical Biology, Vol. 7/Issue 11, Article s, Aug. 15, 2012.
C. Huang et al, “Multivalent nanobodies with rationally optimized linker and valency for intravitreal VEGF neutralization,” Chemical Engineering Science, Volume 270, 15 Apr. 2023, 118521.
S. C. Wu et al., “Engineering Soluble Monomeric Streptavidin with Reversible Biotin Binding Capability,” Protein Structure and Folding, Volume 280, Issue 24, 17 Jun. 2005, Pages 23225-23231.
Y. Ding et al., “‘Ready-to-use’ immunosensor for the detection of small molecules with fast readout,” Biosens. Bioelectron., 2022 Apr. 1:201:113968.
Ding, Y., Li, J., Enterina, J. R., Shen, Y., Zhang, I., Tewson, P. H., . . . Campbell, R. E. (2015). Ratiometric biosensors based on dimerization-dependent fluorescent protein exchange. Nature Methods, 195-198.
Fuller, K. B., Alston, J. M., & Golino, D. A. (2013). The benefits from certified virus-free nursery stock: A case study of grapevine leafroll-3 in the North Coast region of California. Robert Mondavi Institute-Center for Wine Economics, 35.
He et al. “A mix-and-read nanobody-based sandwich homogeneous split-luciferase assay for the rapid detection of human soluble epoxide hydrolase,” Anal. Chem. 2023 April 11; 95(14): 6038-6045. doi:10.1021/acs.analchem.3c00079.
Luker, K. E., Smith, M. C., Luker, G. D., Gammon, S. T., Piwnica-Works, H., & Piwnica-Worms, D. (2004). Kinetics of regulated protein-protein interactions revealed with firefly luciferase complementation imaging in cells and living animals. PNAS, 12288-12293.
Maass, D. R., Sepulveda, J., Pernthaner, A., & Shoemaker, C. B. (2007). Alpaca (Lama pacos) as a convenient source of recombinant camelid heavy chain antibodies (VHHs).
Journal of Immunological Methods, 13-25.
Maree, H. J., Almeida, R. P., Bester, R., Chooi, K. M., Cohen, D., Dolja, V. V., . . . Burger, J. T. (2013). Grapevine leafroll-associated virus 3. Frontiers in Microbiology, 1-21.
Robottom, J. (2018). A Split Enzyme for Biosensors. Leeds: University of Leeds.
van der Linden, R., de Geus, B., Stok, W., Bos, W., van Wassenaar, D., Verrips, T., & Frenken, L. (2000). Induction of immune responses and molecular cloning of the heavy chain antibody repertoire of Lama glama. 185-195.
Yao, Z., Drecun, L., Aboualizadeh, F., Kim, S. J., Li, Z., Wood, H., . . . Stagljar, I. (2021). A homogeneous split-luciferase assay for rapid and and sensitive detection of anti-SARS-CoV-2 antibodies. Nature Communications, 1-8.
WO 2023/235955 A1 (Dec. 14, 2023) to Yao et al.
A S Dixon et al., “NanoLuc Complementation Reporter Optimized for Accurate Measurement of Protein Interactions in Cells,” ACS Chemical Biology, Vol. 11/Issue 2, Nov. 16, 2015.
MP Hall et al., “Engineered Luciferase Reporter from a Deep Sea Shrimp Utilizing a Novel Imidazopyrazinone Substrate,” ACS Chemical Biology, Vol. 7/Issue 11, Article s Aug. 15, 2012.
C. Huang et al., “Multivalent nanobodies with rationally optimized linker and valency for intravitreal VEGF neutralization,” Chemical Engineering Science, Volume 270, 15 Apr. 2023, 118521.
SC Wu et al., “Engineering Soluble Monomeric Streptavidin with Reversible Biotin Binding Capability,” Protein Structure and Folding, Volume 280, Issue 24, 17 Jun. 2005, Pages 23225-23231.
Y Ding et al., ““Ready-to-use” immunosensor for the detection of small molecules with fast readout,” Biosens. Bioelectron., 2022 Apr. 1:201:113968.
Li, D. et al., “Nanobody based Immunoassay for Human Soluble Epoxide Hydrolase Detection using PolyHRP for Signal Enhancement—the Rediscovery of PolyHRP,” Anal. Chem. 2017 May 10; 89(11):6248-6256.
Gransagne M et al., “Development of a highly specific and sensitive VHH-based sandwich immunoassay for the detection of the SARS-CoV-2 nucleoprotein,” J. Biol. Chem., 2022 January; 298(1):101290.
Liu X et al., “Sandwich pair nanobodies, a potential tool for electrochemical immunosensing serum prostate-specific antigen with preferable specificity,” J. Pharmaceutical and Biomedical Analysis, Vol. 158, 2018, Pages 361-369.
J-M Djian et al., “Resistance to grapevine fanleaf virus,” Patent WO2015110601A1, 2015.
Cui Y. et al., “Heavy Chain Single Domain Antibodies to Detect Native Human Soluble Epoxide Hydrolase,” Anal. Bioanal. Chem. 2015 Jul. 31; 407(24):7275-7283.
Inouye, S. & Sasaki, S., “Overexpression, purification and characterization of the catalytic component of Oplophorus luciferase in the deep-sea shrimp, Oplophorus gracilirostris,” J. Protein Expr. Purif 56 (2), 261-268 (2007).
Kornblihtt, A. R. et. al., “Primary structure of human fibronectin: differential splicing may generate at least 10 polypeptides from a single gene,” JOURNAL EMBO. J. 4 (7), 1755-1759 (1985).

1. An assay comprising:
a fusion polypeptide chain comprising:
a first molecule specific for a first target region of a target molecule, the first molecule comprising a first portion of a protein; and
a second molecule specific for a second target region of the target molecule, the second molecule comprising a second portion of the protein; and
one or more linker molecules connected to one or more of the first and the second molecules, the one or more linker molecules comprising one or more of a simple flexible linker and a polypeptide chain; and
wherein, the first and second portions of the protein do not complex with one another and are otherwise nonfunctional in the absence of binding to the target molecule, and
wherein, in the presence of the target molecule, the first and second molecules specific for the first and second target regions of the target molecule recognize and bind to their respective target regions of the target molecule, bringing the first and second portions of the protein into close proximity with one another so as to complex with each other, thus forming a complexed functional protein, and
wherein, the one or more linker molecules possess a property to make the first molecule and the second molecule multivalent when they are fused and made as a fusion protein.

2. The assay of claim 1, wherein the first molecule specific for a first target region of a target molecule further comprises a single-domain antibody (VHH/VNAR).

3. The assay of claim 1, wherein the second molecule specific for a second target region of the target molecule further comprises a single-domain antibody (VHH/VNAR).

4. The assay of claim 1, wherein the protein is an enzyme, fluorescent protein, or polypeptide fragment or variant thereof, that can be split into two or more nonfunctional fragments that can complex with one another when brought into proximity so as to reconstitute a functional protein.

5. The assay of claim 1, wherein the first portion of the protein is an N-terminal fragment of the protein, and the second portion of the protein is a C-terminal fragment of the protein.

6. The assay of claim 1, wherein the one or more linker molecules comprise a flexible peptide or a polypeptide chain that spontaneously forms multimers selected from the group consisting of dimers, trimers, tetramers, or higher-order multimers.

7. The assay of claim 6, wherein the polypeptide linker is selected from streptavidin, alkaline phosphatase, single-stranded DNA-binding protein (SSB), or a functional variant thereof.

8. The assay of claim 6, wherein multimerization of the polypeptide linker provides multivalent binding moieties and/or enzyme fragments, thereby enhancing affinity, specificity, or sensitivity.

9. The assay of claim 1, wherein the fusion protein comprises a binding moiety, a polypeptide linker, and a protein fragment arranged in an order selected from: (a) Binding moiety-Linker-Protein fragment, (b) Linker-Binding moiety-Protein fragment, (c) Binding moiety-Protein fragment-Linker, (d) Binding moiety-Linker Protein Fragment, (e) Linker-Binding moiety-Protein fragment, or (f) Binding moiety-Protein Fragment-Linker.

10.-13. (canceled)

14. The assay of claim 1, wherein the complexed functional protein exhibits spectral features within a UV-visible region, near infrared region, or a combination thereof, either by itself and/or in the presence of its substrate molecule.

15. The assay of claim 1, wherein the protein is selected from the group consisting of luciferase (Luc), aquatic luciferases, β-galactosidase, α-lactamase, β-lactamase, alkaline phosphatase (AP), horseradish peroxidase (HRP), dihydrofolate reductase, fluorescent proteins (FPs), ubiquitin, glucose oxidase, and any combination thereof.

16. The assay of claim 1, wherein the protein comprises a conjugated enzyme.

17. (canceled)

18. The assay of claim 1, wherein the first portion of the protein and the second portion of the protein are configured to catalyze an enzymatic reaction when complexed.

19. The assay of claim 1, wherein the first portion of the protein exhibits low affinity for the second portion of the protein and the first and second portions associated with each other only when brought into close proximity to each other.

20. The assay of claim 1, wherein the protein is configured to be luminescent, fluorescent, colorimetric or a combination thereof either by itself or in presence of its substrate.

21. The assay of claim 1, wherein the assay further comprises a microplate and/or a single tube and a microplate reader and/or a single tube reader capable of capturing the target molecule and reading luminescence or other signals of the assay.

22. The assay of claim 1, wherein the target molecule is selected from a virus, a bacterium, a protein, a peptide, a small molecule, a fungus, a hormone, or any molecular entity that elicits an immune response to produce antibodies.

23. The assay of claim 22, wherein the target molecule is selected from a plant pathogen comprising grapevine leafroll-associated virus type 3 (GLRaV-3) or grapevine fanleaf virus (GVFL), an infectious disease pathogen comprising, SARS-COV-2 nucleocapsid protein, a disease biomarker comprising human prostate-specific antigen (PSA), B-type natriuretic peptide (BNP), or phosphorylated Tau proteins, and a foodborne pathogen comprising Escherichia coli O157, Salmonella spp., or Listeria spp.

24. The assay of claim 1, wherein the assay is performed in a format selected from: (a) a solid-phase assay comprising a capture antibody or nanobody immobilized on a surface; (b) a homogeneous “mix-and-read” assay without immobilization, labeling, or washing; and (c) a lateral flow immunoassay wherein assay reagents are incorporated into a membrane and a signal is detected visually or by instrument.

25. The assay of claim 1, wherein a signal of the complexed functional protein is measured by a microplate reader, handheld luminometer, or lateral flow reader.

26. The assay of claim 25, wherein the signal comprises a spectral feature in a UV-visible, near-infrared, or luminescent region.

27.-51. (canceled)

52. A method of producing a fusion protein according to claim 1, comprising: expressing the fusion protein in a host cell, directing the fusion protein to a periplasmic or extracellular space via a secretion signal peptide, and recovering the fusion protein from a cell culture supernatant for one or more of direct screening and direct use in assays.

53. The method of claim 52, wherein the secretion signal peptide is an OmpA leader peptide.

54. The assay of claim 1, wherein the complexed functional protein provides a signal-to-noise ratio at least three-fold greater than that of a conventional enzyme-linked immunosorbent assay (ELISA).

55. The assay of claim 1, wherein the complexed functional protein provides a signal-to-noise ratio greater than 200 when detecting a target virus.

56. An assay composition comprising:
a first fusion protein comprising a first binding moiety specific for a first epitope of a target analyte, a peptide spacer, a first fragment of a split enzyme, and a thermophilic linker protein selected from single-stranded DNA binding proteins (SSBs) derived from Thermus aquaticus or Thermus thermophilus; and
a second fusion protein comprising a second binding moiety specific for a second epitope of the target analyte, a peptide spacer, a complementary fragment of the split enzyme, and a thermophilic linker protein selected from SSBs derived from T. aquaticus or T. thermophilus;
wherein the enzyme fragments are non-functional when separate and reconstitute into an active enzyme when the first and second binding moieties simultaneously bind the target analyte.

57. A fusion protein comprising:
an N-terminal OmpA leader peptide comprising an amino acid sequence comprising Met-Lys-Lys-Thr-Ala-Ile-Ala-Ile-Ala-Val-Ala-Leu-Ala-Gly-Phe-Ala-Thr-Val-Ala-Gln-Ala (SEQ ID NO:7); and
a protein of interest described herein assuming a form of VHH-spacer-a protein fragment-spacer-polypeptide linker fused to a C-terminus of the N-terminal OmpA leader peptide,
wherein the N-terminal OmpA leader peptide targets the fusion protein for export from a host cell via a Sec pathway,
wherein an inherent permeability of the host cell's outer membrane allows the fusion protein to leak from a periplasmic space into a growth medium, and
wherein the fusion proteins and their combinations can be rapidly screened from a cell culture supernatant.

58. The assay of claim 1, wherein, for detection of grapevine leafroll-associated virus type 3 (GLRaV-3), the assay is at least about 250-fold more sensitive than a conventional ELISA and at least about 80-fold more sensitive than a qPCR or RT-PCR assay.

59. The assay of claim 1, wherein at least one of the first molecule and the second molecule comprises a binding moiety designated as VHH21 specific for grapevine leafroll-associated virus type 3 (GLRaV-3), the binding moiety comprising the amino acid sequence identified in SEQ ID NO. 22.

60. The assay of claim 1, wherein at least one of the first molecule and the second molecule comprises a binding moiety designated as VHH51 specific for grapevine leafroll-associated virus type 3 (GLRaV-3), the binding moiety comprising the amino acid sequence identified in SEQ ID NO. 23.

61. The assay of claim 1, wherein at least one of the first molecule and the second molecule comprises a binding moiety designated as VHH111 specific for grapevine leafroll-associated virus type 3 (GLRaV-3), the binding moiety comprising the amino acid sequence identified in SEQ ID NO. 24.


SEQUENCE LISTING (69 sequences)

SEQ ID NO: 1 - protein, 8 aa, synthetic construct
  REGION 1..8: note = The entire sequence of amino acids 1-8 can be repeated one or more times.
GGGSSGGG

SEQ ID NO: 2 - protein, 5 aa, synthetic construct
  REGION 1..5: note = The entire sequence of amino acids 1-5 can be repeated one or more times.
GGGSS

SEQ ID NO: 3 - protein, 5 aa, synthetic construct
  REGION 1..5: note = The entire sequence of amino acids 1-5 can be repeated one or more times.
GGSSS

SEQ ID NO: 4 - protein, 5 aa, synthetic construct
  REGION 1..5: note = The entire sequence of amino acids 1-5 can be repeated one or more times.
GGGGS

SEQ ID NO: 5 - protein, 159 aa, synthetic construct
  source 1..159: note = N-terminal fragment of nano luciferase (LgLuc)
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 6 - protein, 11 aa, synthetic construct
  source 1..11: note = C-terminal fragment of nano luciferase (SmLuc)
VTGYRLFEEIL

SEQ ID NO: 7 - protein, 21 aa, synthetic construct
  source 1..21: note = OmpA signal peptide
MKKTAIAIAVALAGFATVAQA

SEQ ID NO: 8 - protein, 343 aa, synthetic construct
  source 1..343: note = Fusion 1
AQVQLVESGGGLVQAGGSLRLSCAASGSISSINAMGWYRQAPGKQREVVAETTRGGFIDYADSVKGRFTISRDNAKNTVYLQMNSLKPEDTAVYYCVAAEHGSRQYDYWGQGTQVTVSSGGGSSGGGGSGGGGSGVTGYRLFEEILGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 9 - protein, 345 aa, synthetic construct
  source 1..345: note = Fusion 2
AQVQLVESGGGLVQPGGSLRLSCAASGSIFSIDAWGWYRQAPGKQRELVANIIGGTAEHANSVKGRFTISEDKANNTLYLQMNFLTAEDTAVYYCNIAGRPYRWLDPVDYWGQGTQVTVSSGGGSSGGGGSGGGGSGVTGYRLFEEILGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 10 - protein, 342 aa, synthetic construct
  source 1..342: note = Fusion 3
AQLQLVESGGGLVQAGGSLRLSCAASGPTLSIYNVGWFRQAPGKEREFVARLLWSDGTTYYVDSVKGRFTLSRDYAKNTVYLQMNSLRPEDTAIYSCALDSLGGAPRTWGQGTQVTVSSGGGSSGGGGSGGSGGVTGYRLFEEILGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 11 - protein, 490 aa, synthetic construct
  source 1..490: note = Fusion 4
AQVQLVESGGGLVQAGGSLRLSCAASGSISSINAMGWYRQAPGKQREVVAETTRGGFIDYADSVKGRFTISRDNAKNTVYLQMNSLKPEDTAVYYCVAAEHGSRQYDYWGQGTQVTVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 12 - protein, 492 aa, synthetic construct
  source 1..492: note = Fusion 5
AQVQLVESGGGLVQPGGSLRLSCAASGSIFSIDAWGWYRQAPGKQRELVANIIGGTAEHANSVKGRFTISEDKANNTLYLQMNFLTAEDTAVYYCNIAGRPYRWLDPVDYWGQGTQVTVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 13 - protein, 490 aa, synthetic construct
AQLQLVESGGGLVQAGGSLRLSCAASGPTLSIYNVGWFRQAPGKEREFVARLLWSDGTTYYVDSVKGRFTLSRDYAKNTVYLQMNSLRPEDTAIYSCALDSLGGAPRTWGQGTQVTVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 14 - protein, 152 aa, synthetic construct
AQVQLVESGGGLVQAGGSLRLSCAASGSISSINAMGWYRQAPGKQREVVAETTRGGFIDYADSVKGRFTISRDNAKNTVYLQMNSLKPEDTAVYYCVAAEHGSRQYDYWGQGTQVTVSSGGGSSGGGGSGGGGSGVTGYRLFEEILHHHHHH

SEQ ID NO: 15 - protein, 154 aa, synthetic construct
AQVQLVESGGGLVQPGGSLRLSCAASGSIFSIDAWGWYRQAPGKQRELVANIIGGTAEHANSVKGRFTISEDKANNTLYLQMNFLTAEDTAVYYCNIAGRPYRWLDPVDYWGQGTQVTVSSGGGSSGGGGSGGGGSGVTGYRLFEEILHHHHHH

SEQ ID NO: 16 - protein, 152 aa, synthetic construct
AQLQLVESGGGLVQAGGSLRLSCAASGPTLSIYNVGWFRQAPGKEREFVARLLWSDGTTYYVDSVKGRFTLSRDYAKNTVYLQMNSLRPEDTAIYSCALDSLGGAPRTWGQGTQVTVSSGGGSSGGGGSGGGGSGVTGYRLFEEILHHHHHH

SEQ ID NO: 17 - protein, 299 aa, synthetic construct
AQVQLVESGGGLVQAGGSLRLSCAASGSISSINAMGWYRQAPGKQREVVAETTRGGFIDYADSVKGRFTISRDNAKNTVYLQMNSLKPEDTAVYYCVAAEHGSRQYDYWGQGTQVTVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 18 - protein, 301 aa, synthetic construct
AQVQLVESGGGLVQPGGSLRLSCAASGSIFSIDAWGWYRQAPGKQRELVANIIGGTAEHANSVKGRFTISEDKANNTLYLQMNFLTAEDTAVYYCNIAGRPYRWLDPVDYWGQGTQVTVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 19 - protein, 299 aa, synthetic construct
AQLQLVESGGGLVQAGGSLRLSCAASGPTLSIYNVGWFRQAPGKEREFVARLLWSDGTTYYVDSVKGRFTLSRDYAKNTVYLQMNSLRPEDTAIYSCALDSLGGAPRTWGQGTQVTVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 20 - protein, 183 aa, Streptomyces avidinii
  source 1..183: note = Streptavidin
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 21 - protein, 4 aa, synthetic construct
  REGION 1..4: note = The entire sequence of amino acids 1-4 can be repeated one or more times.
GGGS

SEQ ID NO: 22 - protein, 119 aa, synthetic construct
  source 1..119: note = VHH21
AQVQLVESGGGLVQAGGSLRLSCAASGSISSINAMGWYRQAPGKQREVVAETTRGGFIDYADSVKGRFTISRDNAKNTVYLQMNSLKPEDTAVYYCVAAEHGSRQYDYWGQGTQVTVSS

SEQ ID NO: 23 - protein, 121 aa, synthetic construct
  source 1..121: note = VHH51
AQVQLVESGGGLVQPGGSLRLSCAASGSIFSIDAWGWYRQAPGKQRELVANIIGGTAEHANSVKGRFTISEDKANNTLYLQMNFLTAEDTAVYYCNIAGRPYRWLDPVDYWGQGTQVTVSS

SEQ ID NO: 24 - protein, 119 aa, synthetic construct
  source 1..119: note = VHH111
AQLQLVESGGGLVQAGGSLRLSCAASGPTLSIYNVGWFRQAPGKEREFVARLLWSDGTTYYVDSVKGRFTLSRDYAKNTVYLQMNSLRPEDTAIYSCALDSLGGAPRTWGQGTQVTVSS

SEQ ID NO: 25 - protein, 116 aa, synthetic construct
AQVQLQESGGGLVQPGGSLRLSCAASERMLSINTMGWYRQAPGKQRELVAAITSGGSTNYADSVKGRFTISRENGKNTVYLQMNSLKPEDTAVYYCRWGTDNFNSWGQGTQVTVSS

SEQ ID NO: 26 - protein, 119 aa, synthetic construct
AQVQLVQSGGGSVEAGGSLRLSCVASGLTLSNYRMGWFRQAPGKEREFVASVTWQGSSTHYADSVKGRFTVSRDVAKNTVFLQMNSLKYEDTALYYCAASRWDSGYDYWGQGTQVTVSS

SEQ ID NO: 27 - protein, 119 aa, synthetic construct
EVQLQASGGGLVQAGESLRLSCVVSGGAFRIMDMGWFRQVPGKQREVVGVISSGGYTNYADSVKGRFTISRDNAKRTVYLHMNSLKPEDTAVYYCNIIPKSDQGAVNTWGKGTLVSVSS

SEQ ID NO: 28 - protein, 126 aa, synthetic construct
EVQLQASGGGLMQAGGSLSLSCAASGRTDSTQHMAWFRQAPGKEREFVTAIQWRGGGTYYADSVKGRFTISRDNAKNTVYLQMNSLKPEDTAVYYCATNTRWTYFSPTVPDRYDYWGQGTQVTVSS

SEQ ID NO: 29 - protein, 131 aa, synthetic construct
QVQLQESGGGSVQVGGSLRLACAASGDTFSGYLAAWFRQAPGKEREGVAAINSVRHTTSYANSVKGRFTISKDNADNMMYLEMNSLKPEDTAIYYCAAADAIGLAEYWSTPTLSAARYKYWGQGTQVTVSS

SEQ ID NO: 30 - protein, 131 aa, synthetic construct
QVQLQESGGGAVQPGGSLKLSCEASGDVPENGYMAWFRQAPGKEREGVAAINSVRHTTSYADSVKGRFTISKDNADNMMYLEMNSLKPEDTAIYYCAAADAIGLAEYWSTPTLSAARYKYWGQGTQVTVSS

SEQ ID NO: 31 - protein, 126 aa, synthetic construct
QVQLVESGGGLVQPGGSLRLSCAASGFTLDDYVMGWFRQAPGKEREGVSCISTSDGRTYYADSVKGRFTISRDNAKNTLYLQMNSLKPEDTAVYYCAALRTPYITYLITPSEYDYWGQGTQVTVSA

SEQ ID NO: 32 - protein, 116 aa, synthetic construct
QVQLVESGGGLVQAGGSLRLSCAASGSIFSIISMNWYRQAPGKQREWVATIYSGNLTDYADSVKGRFTISRDNAKNTVYLQMNTLKPEDTAVYYCNAESGSRAGYWGQGTQVTVAS

SEQ ID NO: 33 - protein, 196 aa, synthetic construct
MAYSTLFIIALTAVVTQASSTQKSNLTFTLADFVGDWQQTAGYNQDQVLEQGGLSSLFQALGVSVTPIQKVVLSGENGLKADIHVIIPYEGLSGFQMGLIEMIFKVVYPVDDHHFKIILHYGTLVIDGVTPNMIDYFGRPYPGIAVFDGKQITVTGTLWNGNKIYDERLINPDGSLLFRVTINGVTGWRLCENILA

SEQ ID NO: 34 - protein, 275 aa, Tateyamaria sp.
MNEMLRNQPVLGMFNAYMIRNDKSGAAMLGFLTNTKVTGYKLFEEFLSKSAKTPIALDGKQQKAVDDVMRGPQKDAAAGLNKLVPGLKKACLTYLDKKAIPAFYKTKDKPGSVFYQHCRPHAEKNCEGRFGKIAVATKRLGLTDQMLVKEIMVQLYMGNNKSAAAAATKAAKKAGLKMPAVVIVDAVERQRGLIGYHDVKIDAKSLVFCGFQNVNDKDILKLMKQMVEHHYDKKDSKAKKAFEQIKKLEPKSSPITKLKYDAFIKLLKKKGTITT

SEQ ID NO: 35 - protein, 11 aa, synthetic construct
VTGYKLFEEFL

SEQ ID NO: 36 - protein, 421 aa, synthetic construct
MKKLYLLMLVIGLVLVNACGSDDNGNGTDSESPSAPLNLIASNVSDTSLELSWTASTDNTAVTGYRLYEEISGNVTPLGGSTTTYMVTGLTPSTPYKFYVTAIDAAGNESSQSNTVEISTDEAPLEFLTNLSEMGIFTGDLVNLEPAENVQLYELNSTLFTDYAAKQRLIRFPEGQAMRYNNSDQFPVFPDNTLMAKTFFYYINDQDPGQGKQIIETRLLLKIEGAWQVGNYVWNASQTEATYRETGSEIPISYIDGNGDTQNVDYQIPSKADCIICHSNSNTIIPIGPKLRTMNFVPSYTNMNQLEYFKANGLLEGLGSASSISVLPDWTNDVLYTLEERARGYIDINCAHCHQPGGAVTNFNIDFQYETPYADTGIYPNRGEIEMRIQSTLPSYRMPQLGRTVVHEEAVAVLIAYLDTL

SEQ ID NO: 37 - protein, 11 aa, synthetic construct
VTGYRLYEEIS

SEQ ID NO: 38 - protein, 491 aa, synthetic construct
EVQLQASGGGLVQAGESLRLSCVVSGGAFRIMDMGWFRQVPGKQREVVGVISSGGYTNYADSVKGRFTISRDNAKRTVYLHMNSLKPEDTAVYYCNIIPKSDQGAVNTWGKGTLVSVSSGGGSSGGGGSGGGGSGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 39 - protein, 497 aa, synthetic construct
EVQLQASGGGLMQAGGSLSLSCAASGRTDSTQHMAWFRQAPGKEREFVTAIQWRGGGTYYADSVKGRFTISRDNAKNTVYLQMNSLKPEDTAVYYCATNTRWTYFSPTVPDRYDYWGQGTQVTVSSGGGSSGGGGSGGGGSGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHH

SEQ ID NO: 40 - protein, 299 aa, synthetic construct
EVQLQASGGGLVQAGESLRLSCVVSGGAFRIMDMGWFRQVPGKQREVVGVISSGGYTNYADSVKGRFTISRDNAKRTVYLHMNSLKPEDTAVYYCNIIPKSDQGAVNTWGKGTLVSVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 41 - protein, 306 aa, synthetic construct
EVQLQASGGGLMQAGGSLSLSCAASGRTDSTQHMAWFRQAPGKEREFVTAIQWRGGGTYYADSVKGRFTISRDNAKNTVYLQMNSLKPEDTAVYYCATNTRWTYFSPTVPDRYDYWGQGTQVTVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 42 - protein, 343 aa, synthetic construct
EVQLQASGGGLVQAGESLRLSCVVSGGAFRIMDMGWFRQVPGKQREVVGVISSGGYTNYADSVKGRFTISRDNAKRTVYLHMNSLKPEDTAVYYCNIIPKSDQGAVNTWGKGTLVSVSSGGGSSGGGGSGGGGSGVTGYRLFEEILGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 43 - protein, 350 aa, synthetic construct
EVQLQASGGGLMQAGGSLSLSCAASGRTDSTQHMAWFRQAPGKEREFVTAIQWRGGGTYYADSVKGRFTISRDNAKNTVYLQMNSLKPEDTAVYYCATNTRWTYFSPTVPDRYDYWGQGTQVTVSSGGGSSGGGGSGGGGSGVTGYRLFEEILGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 44 - protein, 151 aa, synthetic construct
EVQLQASGGGLVQAGESLRLSCVVSGGAFRIMDMGWFRQVPGKQREVVGVISSGGYTNYADSVKGRFTISRDNAKRTVYLHMNSLKPEDTAVYYCNIIPKSDQGAVNTWGKGTLVSVSSGGGSSGGGGSGGSGGVTGYRLFEEILHHHHHH

SEQ ID NO: 45 - protein, 158 aa, synthetic construct
EVQLQASGGGLMQAGGSLSLSCAASGRTDSTQHMAWFRQAPGKEREFVTAIQWRGGGTYYADSVKGRFTISRDNAKNTVYLQMNSLKPEDTAVYYCATNTRWTYFSPTVPDRYDYWGQGTQVTVSSGGGSSGGGGSGGSGGVTGYRLFEEILHHHHHH

SEQ ID NO: 46 - protein, 488 aa, synthetic construct
AQVQLQESGGGLVQPGGSLRLSCAASERMLSINTMGWYRQAPGKQRELVAAITSGGSTNYADSVKGRFTISRENGKNTVYLQMNSLKPEDTAVYYCRWGTDNFNSWGQGTQVTVSSGGGSSGGGGSGGGGSGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 47 - protein, 491 aa, synthetic construct
AQVQLVQSGGGSVEAGGSLRLSCVASGLTLSNYRMGWFRQAPGKEREFVASVTWQGSSTHYADSVKGRFTVSRDVAKNTVFLQMNSLKYEDTALYYCAASRWDSGYDYWGQGTQVTVSSGGGSSGGGGSGGGGSGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 48 - protein, 296 aa, synthetic construct
AQVQLQESGGGLVQPGGSLRLSCAASERMLSINTMGWYRQAPGKQRELVAAITSGGSTNYADSVKGRFTISRENGKNTVYLQMNSLKPEDTAVYYCRWGTDNFNSWGQGTQVTVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 49 - protein, 299 aa, synthetic construct
AQVQLVQSGGGSVEAGGSLRLSCVASGLTLSNYRMGWFRQAPGKEREFVASVTWQGSSTHYADSVKGRFTVSRDVAKNTVFLQMNSLKYEDTALYYCAASRWDSGYDYWGQGTQVTVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 50 - protein, 340 aa, synthetic construct
AQVQLQESGGGLVQPGGSLRLSCAASERMLSINTMGWYRQAPGKQRELVAAITSGGSTNYADSVKGRFTISRENGKNTVYLQMNSLKPEDTAVYYCRWGTDNFNSWGQGTQVTVSSGGGSSGGGGSGGGGSGVTGYRLFEEILGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 51 - protein, 343 aa, synthetic construct
AQVQLVQSGGGSVEAGGSLRLSCVASGLTLSNYRMGWFRQAPGKEREFVASVTWQGSSTHYADSVKGRFTVSRDVAKNTVFLQMNSLKYEDTALYYCAASRWDSGYDYWGQGTQVTVSSGGGSSGGGGSGGGGSGVTGYRLFEEILGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 52 - protein, 148 aa, synthetic construct
AQVQLQESGGGLVQPGGSLRLSCAASERMLSINTMGWYRQAPGKQRELVAAITSGGSTNYADSVKGRFTISRENGKNTVYLQMNSLKPEDTAVYYCRWGTDNFNSWGQGTQVTVSSGGGSSGGGGSGGSGGVTGYRLFEEILHHHHHH

SEQ ID NO: 53 - protein, 151 aa, synthetic construct
AQVQLVQSGGGSVEAGGSLRLSCVASGLTLSNYRMGWFRQAPGKEREFVASVTWQGSSTHYADSVKGRFTVSRDVAKNTVFLQMNSLKYEDTALYYCAASRWDSGYDYWGQGTQVTVSSGGGSSGGGGSGGSGGVTGYRLFEEILHHHHHH

SEQ ID NO: 54 - protein, 503 aa, synthetic construct
QVQLQESGGGSVQVGGSLRLACAASGDTFSGYLAAWFRQAPGKEREGVAAINSVRHTTSYANSVKGRFTISKDNADNMMYLEMNSLKPEDTAIYYCAAADAIGLAEYWSTPTLSAARYKYWGQGTQVTVSSGGGSSGGGGSGGGGSGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 55 - protein, 503 aa, synthetic construct
QVQLQESGGGAVQPGGSLKLSCEASGDVPENGYMAWFRQAPGKEREGVAAINSVRHTTSYADSVKGRFTISKDNADNMMYLEMNSLKPEDTAIYYCAAADAIGLAEYWSTPTLSAARYKYWGQGTQVTVSSGGGSSGGGGSGGGGSGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 56 - protein, 311 aa, synthetic construct
QVQLQESGGGSVQVGGSLRLACAASGDTFSGYLAAWFRQAPGKEREGVAAINSVRHTTSYANSVKGRFTISKDNADNMMYLEMNSLKPEDTAIYYCAAADAIGLAEYWSTPTLSAARYKYWGQGTQVTVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 57 - protein, 311 aa, synthetic construct
QVQLQESGGGAVQPGGSLKLSCEASGDVPENGYMAWFRQAPGKEREGVAAINSVRHTTSYADSVKGRFTISKDNADNMMYLEMNSLKPEDTAIYYCAAADAIGLAEYWSTPTLSAARYKYWGQGTQVTVSSGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 58 - protein, 355 aa, synthetic construct
QVQLQESGGGSVQVGGSLRLACAASGDTFSGYLAAWFRQAPGKEREGVAAINSVRHTTSYANSVKGRFTISKDNADNMMYLEMNSLKPEDTAIYYCAAADAIGLAEYWSTPTLSAARYKYWGQGTQVTVSSGGGSSGGGGSGGGGSGVTGYRLFEEILGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 59 - protein, 355 aa, synthetic construct
QVQLQESGGGAVQPGGSLKLSCEASGDVPENGYMAWFRQAPGKEREGVAAINSVRHTTSYADSVKGRFTISKDNADNMMYLEMNSLKPEDTAIYYCAAADAIGLAEYWSTPTLSAARYKYWGQGTQVTVSSGGGSSGGGGSGGGGSGVTGYRLFEEILGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 60 - protein, 163 aa, synthetic construct
QVQLQESGGGSVQVGGSLRLACAASGDTFSGYLAAWFRQAPGKEREGVAAINSVRHTTSYANSVKGRFTISKDNADNMMYLEMNSLKPEDTAIYYCAAADAIGLAEYWSTPTLSAARYKYWGQGTQVTVSSGGGSSGGGGSGGSGGVTGYRLFEEILHHHHHH

SEQ ID NO: 61 - protein, 163 aa, synthetic construct
QVQLQESGGGAVQPGGSLKLSCEASGDVPENGYMAWFRQAPGKEREGVAAINSVRHTTSYADSVKGRFTISKDNADNMMYLEMNSLKPEDTAIYYCAAADAIGLAEYWSTPTLSAARYKYWGQGTQVTVSSGGGSSGGGGSGGSGGVTGYRLFEEILHHHHHH

SEQ ID NO: 62 - protein, 498 aa, synthetic construct
QVQLVESGGGLVQPGGSLRLSCAASGFTLDDYVMGWFRQAPGKEREGVSCISTSDGRTYYADSVKGRFTISRDNAKNTLYLQMNSLKPEDTAVYYCAALRTPYITYLITPSEYDYWGQGTQVTVSAGGGSSGGGGSGGGGSGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 63 - protein, 488 aa, synthetic construct
QVQLVESGGGLVQAGGSLRLSCAASGSIFSIISMNWYRQAPGKQREWVATIYSGNLTDYADSVKGRFTISRDNAKNTVYLQMNTLKPEDTAVYYCNAESGSRAGYWGQGTQVTVASGGGSSGGGGSGGGGSGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 64 - protein, 306 aa, synthetic construct
QVQLVESGGGLVQPGGSLRLSCAASGFTLDDYVMGWFRQAPGKEREGVSCISTSDGRTYYADSVKGRFTISRDNAKNTLYLQMNSLKPEDTAVYYCAALRTPYITYLITPSEYDYWGQGTQVTVSAGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 65 - protein, 296 aa, synthetic construct
QVQLVESGGGLVQAGGSLRLSCAASGSIFSIISMNWYRQAPGKQREWVATIYSGNLTDYADSVKGRFTISRDNAKNTVYLQMNTLKPEDTAVYYCNAESGSRAGYWGQGTQVTVASGGGSSGGGGSGGSGGMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 66 - protein, 350 aa, synthetic construct
QVQLVESGGGLVQPGGSLRLSCAASGFTLDDYVMGWFRQAPGKEREGVSCISTSDGRTYYADSVKGRFTISRDNAKNTLYLQMNSLKPEDTAVYYCAALRTPYITYLITPSEYDYWGQGTQVTVSAGGGSSGGGGSGGGGSGVTGYRLFEEILGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 67 - protein, 340 aa, synthetic construct
QVQLVESGGGLVQAGGSLRLSCAASGSIFSIISMNWYRQAPGKQREWVATIYSGNLTDYADSVKGRFTISRDNAKNTVYLQMNTLKPEDTAVYYCNAESGSRAGYWGQGTQVTVASGGGSSGGGGSGGGGSGVTGYRLFEEILGGGSSGGGMRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQHHHHHH

SEQ ID NO: 68 - protein, 158 aa, synthetic construct
QVQLVESGGGLVQPGGSLRLSCAASGFTLDDYVMGWFRQAPGKEREGVSCISTSDGRTYYADSVKGRFTISRDNAKNTLYLQMNSLKPEDTAVYYCAALRTPYITYLITPSEYDYWGQGTQVTVSAGGGSSGGGGSGGSGGVTGYRLFEEILHHHHHH

SEQ ID NO: 69 - protein, 148 aa, synthetic construct
QVQLVESGGGLVQAGGSLRLSCAASGSIFSIISMNWYRQAPGKQREWVATIYSGNLTDYADSVKGRFTISRDNAKNTVYLQMNTLKPEDTAVYYCNAESGSRAGYWGQGTQVTVASGGGSSGGGGSGGSGGVTGYRLFEEILHHHHHH